(19) United States
(12) Patent Application Publication
(10) Pub. No.: US 2023/0223151 A1
(43) Pub. Date: Jul. 13, 2023

(54) IDENTIFYING AN INDIVIDUAL'S LIKELIHOOD OF HAVING AN ACUTE HEART FAILURE

(71) Applicant: THE UNIVERSITY COURT OF THE UNIVERSITY OF EDINBURGH, Edinburgh (GB)

(72) Inventors: Dimitrios DOUDESIS, Edinburgh (GB); Kuan Ken LEE, Edinburgh (GB); Nicholas Linton MILLS, Edinburgh (GB)

(21) Appl. No.: 18/009,444

(22) PCT Filed: Jun. 11, 2021
(86) PCT No.: PCT/GB2021/051470

§ 371 Date: Dec. 9, 2022

(30) Foreign Application Priority Data
Jun. 12, 2020 (GB) 2008994.2

(51) Int. Cl. G16H 50/30; G06N 20/20; G16H 50/20

(52) U.S. Cl. CPC G16H 50/30; G06N 20/20; G16H 50/20

(57) ABSTRACT

There is provided a method, systems and device to provide an indication of the probability of acute heart failure in a subject/individual. Suitably a device, systems and methods to determine a likelihood score based upon the concentration of natriuretic peptides in blood and at least two other clinical parameters. The method of determining acute heart failure can comprise the steps of combining the level of natriuretic peptide in a sample from an individual with at least two other clinical parameters from the individual in a statistical model to compute the probability of acute heart failure for the individual patient wherein the level of natriuretic peptide is provided as a continuous variable in the model.

### FIELD OF THE INVENTION

The invention provides a method to provide an indication of the probability of acute heart failure in a subject/individual. This can be used as a decision-support tool using natriuretic peptide concentrations, for example N-terminal pro-B-type natriuretic peptide (NT-proBNP), B-type natriuretic peptide (BNP) and mid-regional pro-atrial natriuretic peptide (MR-proANP), and simple, objective clinical variables. In particular, there is provided systems and methods to determine a likelihood score based upon the concentration of natriuretic peptides in blood and at least two other clinical parameters selected from a group comprising, age, sex, previous history of heart failure, body mass index, renal dysfunction, anaemia, COPD, diastolic blood pressure, systolic blood pressure, mean arterial pressure, heart rate and diabetes mellitus. The likelihood score can then be utilised to stratify subjects to allow them to be ruled in or out of a diagnostic group or to select particular treatment(s) or tests that the physician considers most suitable.

### BACKGROUND

Over 6.2 million people are currently living with heart failure in the US alone, and together they make over 1.1 million visits to the Emergency Department per annum. The accurate and timely diagnosis of acute heart failure can be challenging, and therefore both national and international guidelines recommend natriuretic peptide testing to aid in the diagnosis.

NT-proBNP is known to be released in heart failure. At present it is used in the assessment of chronic heart failure, but its use in acute heart failure has been difficult to implement as a normal level in one person could be an abnormal level in another. NT-proBNP testing has been indicated to aid in the evaluation of patients with suspected acute heart failure, with a recent study-level meta-analysis reporting that the guideline recommended NT-proBNP threshold of 300 pg/mL has excellent performance to exclude acute heart failure. However, ruling in heart failure with NT-proBNP is known to be more challenging (Ponikowski P, Voors A A, Anker S D, et al. 2016 ESC Guidelines for the diagnosis and treatment of acute and chronic heart failure. European Heart Journal 2016; 37:2129-200; National Institute for Health and Care Excellence, NICE Clinical Guideline 187 CG187]. Acute Heart Failure. 2014; Yancy C W, Jessup M, Bozkurt B, et al. 2017 ACC/AHA/HFSA Focused Update of the 2013 ACCF/AHA Guideline for the Management of Heart Failure: A Report of the American College of Cardiology/American Heart Association Task Force on Clinical Practice Guidelines and the Heart Failure Society of America. Circulation 2017; 136:e137-e61; Roberts E, Ludman A J, Dworzynski K, et al. The diagnostic accuracy of the natriuretic peptides in heart failure: systematic review and diagnostic meta-analysis in the acute care setting. BMJ 2015; 350:h910). Alternative approaches including the use of age-specific NT-proBNP thresholds have been proposed (Januzzi J L, Jr., Chen-Tournoux A A, Christenson R H, et al. N-Terminal Pro-B-Type Natriuretic Peptide in the Emergency Department: The ICON-RELOADED Study. J Am Coll Cardiol 2018; 71:1191-200).

Studies such as Khanam et al “Validation of the MAGGIC (Meta-Analysis Global Group in Chronic Heart Failure) heart failure score and the effect of adding natriuretic peptide for predicting mortality after discharge in hospitalized patients with heart failure”, PLoS ONE 13(11) relate to Chronic Heart Failure rather than Acute Heart Failure. Moreover, this score predicts mortality rather than diagnosis (a different clinical outcome).

WO2013/120114 is also directed to predicting an adverse effect rather than providing a tool directed to diagnosis.

WO2004034902 is directed towards chronic heart failure and discusses the combination of combination of measuring a biomarker and conducting an ECG measurement.

WO2008039931 is directed towards the use of an algorithmic scoring method for the diagnosis, prognosis and validation risk stratification of dyspnoeic patients who may or may not suffer from acute congestive heart failure. This scoring method utilised age stratified levels of BNP and or NT-proBNP.

US2015199491 relates to chronic rather than acute heart failure and uses parameter thresholds as determination of development of heart failure rather than providing a support tool for diagnosis of acute heart failure.

Typically, current approaches are based on thresholds selected to give good negative and positive predictive value; however, the optimal method to utilise natriuretic peptides is uncertain and improved methods in this regard are required.

### SUMMARY OF THE INVENTION

The inventors have utilised models to consider the level of natriuretic peptide in combination with clinical characteristics to provide a probability score for acute heart failure for an individual patient. The inventors have determined that natriuretic peptide, for example B-type natriuretic peptide (BNP) and its pro-fragment, N-terminal pro-B-type natriuretic (NT-proBNP) and mid-regional pro-atrial natriuretic peptide (MR-proANP) can be provided using a continuous function to provide an improved probability score for acute heart failure. Unexpectedly, the NPV of NT-proBNP at the guideline recommended threshold to rule-out acute heart failure was lower than previous estimates. In particular, the NPV was substantially lower in older patients, and those with obesity or prior heart failure, where the false negative rates with conventional thresholds were as high as one in five.

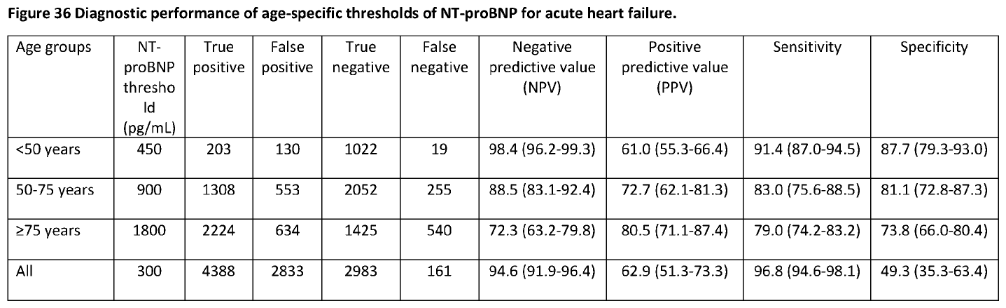

Age-stratified thresholds have performed well to rule-in the diagnosis of acute heart failure in certain circumstances. However, the PPV at these thresholds did not give equivalent performance across different age groups. The PRIDE score, as discussed in the art, uses the age stratified thresholds for NT-ProBNP to ensure that the diagnostic performance of the score to rule out and rule in acute heart failure is similar in patients above 50 years (900 pg/mL threshold) and below 50 years (450 pg/mL threshold). These thresholds did not perform consistently in meta-analysis undertaken by the inventors giving a NPV of 98.4 and 88.5, and a PPV of 61.0 and 72.3 in those patients less than and greater than 50 years old, respectively (FIG. 36). In contrast, using the methods of the invention, the score (the CoDE-HF score) of 4.2 to rule out acute heart failure gave a NPV of 99.4 and 98.7 in those below and above 50 years, and a score of 53.4 to rule in acute heart failure gave a PPV of 77.3 and 76.5 in those below and above 50 years in the inventor's external validation cohort.”

A single threshold approach or the use of thresholds in isolation when natriuretic peptide, in particular NT-ProBNP, was determined to be influenced by many factors and co-morbidities, was considered by the inventors to be disadvantageous.

To improve the clinical utility of natriuretic peptide, the inventors have developed and validated a clinical decision-support tool, and a method to generate a score, which incorporates at least one natriuretic peptide, for example at least one of NT-proBNP, BNP and MR-proANP as a continuous measure in combination with other simple, objective clinical variables to provide an individualized assessment of the likelihood of the diagnosis of acute heart failure

The invention provides a method of identifying an individual's likelihood of having acute heart failure comprising the steps of

(a) providing the level of natriuretic peptide in a sample from the individual and

(b) combining the level of natriuretic peptide with at least two other clinical parameters in a statistical model to compute the probability of acute heart failure (e.g. score of 0-100) for the individual patient.

Suitably the statistical model may be selected from generalised linear mixed model [GLMM] and extreme gradient boosting machine learning algorithm [XGBoost]). Suitably the model may utilise natriuretic peptide concentration as a continuous measure. i.e. wherein the natriuretic peptide level or natriuretic peptide concentration is not provided as a segmented value as high, medium or low and/or relative to a threshold provided by a single variable such as age. Suitably the algorithm generated by the GLMM and/or XGBoost models allows the consideration of a continuous natriuretic peptide value in combination with the at least two other clinical parameters.

The clinical parameters include, but are not limited to, at least two of the following: age, renal function for example via creatinine or eGFR levels, haemoglobin, body mass index, heart rate, blood pressure-for example diastolic blood pressure, systolic blood pressure, mean arterial pressure, -peripheral oedema, prior history of heart failure, chronic obstructive pulmonary disease, ischaemic heart disease, and diabetes mellitus.

Suitably, renal function may be measured by estimated glomerular filtration rate, creatinine clearance rate or serum/plasma creatinine.

Suitably, body mass index may be represented by the use of two or more categories of underweight, normal weight, overweight or obese.

In embodiments of the invention, individual clinicians or healthcare providers have the option to select different low or high-probability scores as thresholds for clinical decision making within care pathways where the diagnostic performance (sensitivity, specificity, positive predictive value and negative predictive value) is more suited to the local setting. Suitably a rule-out threshold that achieves a negative predicted value (NPV) of 98% and sensitivity of 90% and a rule-in threshold that achieves positive predicted value (PPV) of 75% and specificity of 90% may be utilised. ROC curve analysis is used to determine the cut-off point for the diagnosis of acute heart failure. As would be understood by one of skill in the art, the ROC curve plots a variables sensitivity—true positive fraction, against specificity (false positive). The ROC curve can be used to establish the optimum probability/weighting for a parameter to provide a positive predictive value in view of a cutoff selected by the clinician/or provided in the computer tool or software. These methods are well known in the art.

As would be understood a true positive is a where the patient is considered to be positive according to the method of the invention and also has a confirmed diagnosis of acute heart failure. A false positive is where the patient is considered to be positive according to the method of the invention, but does not have a diagnosis of acute heart failure. A false negative is a patient which does have acute heart failure, but is failed to be recognised by the method of the invention. A true negative is a patient that does not have acute heart failure and is indicated as being negative by the method of the invention. Sensitivity means the probability of the method of the invention providing a positive result when the patient does have acute heart failure. Specificity is the probability the method of the invention provides a negative result when the patient does not have acute heart failure. NPV is the probability that an individual diagnosed as not having acute heart failure. This can be calculated as the number of true negatives divided by the sum of true negatives and false negatives. PPV means the probability that an individual diagnosed as having acute heart failure actually has the condition.

In logistic regression, the logistic function computes probabilities that are linear on the logit scale:

$$z = Xw$$

$$P ( y = 1 \vert X ) = \frac{1}{1 + exp ( - z )}$$

Unlike logistic regression, in the extreme gradient boosting machine learning algorithm [XGBoost]), the parameters in X are constructed as the terminal nodes of an ensemble of decision trees using the boosting procedure. Each row of X collects the terminal leaves for each sample; the row is a T-hot binary vector, for T the number of trees.

There are n columns in X, one column for each terminal node. There is no expression for the total number of terminal nodes, because the number of nodes can vary between trees.

Each leaf in the tree has an associated “weight.” That weight is recorded in w. To be conformable with X, there are n elements in w. The weights themselves are derived from the gradient boosting procedure.

For each different patient, the parameters considered will be assigned different individual weightings to provide a score. The weighted sum of for the total number of terminal nodes can provide a diagnostic score for a patient. ROC curves to calculate the rule in and rule out thresholds.

Suitably, the method may be provided in a computer based tool through which a clinician can input data, or wherein the computer based tool can receive data to allow establishment or the ruling out of acute heart failure. The computer based tool can provide a suggestion as to the way in which the clinician should interpret and/or use the score. For example, the computer based tool may provide treatment or care recommendations. Suitably the computer based tool can be provided in software, hardware or a combination of both, for example an app which may be provided on a device such as a phone or other digital device having one or more processors. Suitably the computer based tool may comprise memory or other data storage to allow a computer program to be provided. Suitably the memory or data storage may comprise subject or patient related data that may be used to provide clinical parameters for the method. Suitably the computer based tool may be able to communicate with an external device, for example a sensor to measure a clinical parameter or a device to provide a level of natriuretic peptide, for example at least one of NT-proBNP, BNP and MR-proANP or a combination of the same. Suitably the computer based tool is capable of providing a signal indicative of the status of acute heart failure in an individual. Suitably the signal may display a numerical score to a user indicative of mortality. Suitably the signal may display a score which is a predictor of heart failure. Suitably the signal may display a score which is a predictor of mortality in a period of time, for example one year.

Suitably, additional clinical parameters, for example systolic blood pressure, diastolic blood pressure, mean arterial pressure, heart rate, haemoglobin, renal function, ECG data, cardiac biomarker concentration e.g. troponin concentration or another biomarker, may also be included in the method.

Suitably the clinical parameters may be assessed at a single point in time, for example based on single blood sample.

The invention further provides a system to identify an individual's likelihood of having acute heart failure, the system comprising a computer processor, memory comprising one or more computer programs wherein one or more of the computer programs comprise a statistical model to compute the probability of acute heart failure (e.g. score of 0-100) for an individual patient by combining the level of natriuretic peptide of the individual with at least two other clinical parameters from the individual. Suitably there is provided a system in a handheld device such as a smartphone. Suitably the method may be provided as part of a smartphone app. Suitably the system has an algorithm provided in the device by incorporation of software or the means to receive a result as calculated by an algorithm remotely from the device. Suitably the system can comprise a device for measuring natriuretic peptide. Suitably the system can comprise a device for measuring natriuretic peptide and at least another, suitably at least two other clinical parameters.

Two statistical models were developed (generalised linear mixed model [GLMM] and extreme gradient boosting machine learning algorithm [XGBoost]). Both models utilised natriuretic peptide concentrations as a continuous measure and at least two other objective clinical variables that are known to be associated with acute heart failure (for example age, renal function, haemoglobin, body mass index, heart rate, blood pressure, for example systolic blood pressure, diastolic blood pressure, and/or mean arterial pressure, ECG data, cardiac troponin concentration, peripheral oedema, prior history of heart failure, chronic obstructive pulmonary disease, ischaemic heart disease and diabetes mellitus). The clinical variables can be predefined simple parameters that can be easily measured.

In the present invention, natriuretic peptide concentrations are provided as a continuous measure directly from the laboratory or physiological measurement without segregation into discrete groups or threshold values.

In contrast to the conventional thresholds as utilised in relation to NTproBNP and age (three particular threshold values utilised), utilising a continuous measure of natriuretic peptide, the inventors have determined that they can utilise other factors (in addition or alternatively to age) that influence natriuretic levels. As would be understood, using conventional methods, it is not possible to stratify levels based on all the permutations of for example 10 variables. The method proposed by the present inventors enables multiple additional factors, for example at least two, at least 3, at least 4, at least 5, at least 6, at least 7, at least 8, at least 9, at least 10 factors to be taken into account when considering the value of natriuretic peptide, in relation to the probability of acute heart failure. In particular the method provided herein takes the multiple variables into account and presents it as a result in a simple form that can be easily applied into clinical practice.

To account for missing data in the GLMM model, joint-modelling multiple imputation with random study specific covariance matrices fitted with a Markov chain Monte Carlo algorithm was used. A logarithmic transformation of natriuretic peptide concentrations was used in the model to account for the positive-skew in natriuretic peptide concentrations. Non-linear relationships between continuous variables and the diagnosis of acute heart failure were evaluated using multivariable fractional polynomial methods.

Extreme Gradient Boosting (XGBoost) is a supervised machine learning technique proposed by Chen and Guestrin (Chen T, Guestrin C. XGBoost: A Scalable Tree Boosting System. ArXiv e-prints 2016). In brief, gradient boosting employs an ensemble technique to iteratively improve model accuracy for regression and classification problems. This is achieved by creating sequential models, using decision trees as learners where subsequent models attempt to correct errors of the preceding models. XGBoost refers to the re-engineering of gradient boosting to significantly improve the speed of the algorithm by pushing the limits of computational resources.

The mathematical formula for the gradient boosting model can be described as:

$${\hat{y}}_{i} = {\sum }_{k = 1}^{K} {f}_{k} ( {x}_{i} ) , {f}_{k} ∈ F \qquad ( 1 )$$

where f is an function that map each variable vector xᵢ (xᵢ={xᵢ, x₂, . . . , xₙ}, i=1, 2, N) to the outcome yᵢ K is the number of Classification and Regression Trees (CART) and F is the space of function containing all CART.⁴

XGBoost optimises an objective function of the form:

$$Obj = {\sum }_{i = 1}^{N} l ( {y}_{i} , {\hat{y}}_{i} ) + {\sum }_{k = 1}^{K} Ω ( {f}_{k} ) \qquad ( 2 )$$

Where the first term is a loss function, which evaluates how well the model fits the data by measuring the difference between the prediction ŷᵢ and the outcome yᵢ. The second term, the regularization term, is used by XGBoost to avoid overfitting by penalizing the complexity of the model. Furthermore, to improve and fully leverage the advantages of XGBoost the inventors tuned the hyper-parameters of the algorithm through a grid search strategy using 10-fold cross-validation. The algorithm was developed using the R package ‘xgboost’ (https://cran.r-project.org/web/packages/xgboost/).

For both GLMM and XGBoost models, ten iterations of 10-fold cross-validation can be performed to generate the probability score for each patient. The score that would classify the highest proportion of patients as high- or low-probability of acute heart failure with predefined optimal diagnostic performance to rule-in and rule-out acute heart failure was then computed.

The invention may further provide a method of identifying an individual's likelihood of having acute heart failure comprising the steps of

(a) obtaining the level of natriuretic peptide in a sample from the individual and

(b) obtaining values for least two other clinical factors such as age, renal function, haemoglobin, body mass index, heart rate, blood pressure, for example systolic blood pressure, diastolic blood pressure, and/or mean arterial pressure, ECG data, cardiac biomarker concentration, peripheral oedema, prior history of heart failure, chronic obstructive pulmonary disease, ischaemic heart disease and diabetes mellitus for example from an electronic record for the individual and assigning a probability score of acute heart failure to an individual based on a statistical model to compute the probability of acute heart failure (score of 0-100) for an individual patient.

Suitably the two other clinical factors may be selected from a list comprising or consisting of age, renal function, haemoglobin, body mass index, heart rate, blood pressure, for example systolic blood pressure, diastolic blood pressure, and/or mean arterial pressure, ECG data and cardiac biomarker concentration, peripheral oedema, prior history of heart failure, chronic obstructive pulmonary disease, ischaemic heart disease and diabetes mellitus.

As would be understood by those of skill in the art, values for use in the method may be entered by a clinician themselves, or by support to the clinician, into a system of the invention, for example a smartphone app if the variables are not readily available from electronic healthcare records. If all, or a portion of the required variables are available on an electronic record for a subject, then the score can be determined by the system, for example the app, operating directly within the electronic healthcare record.

Suitably an electronic record may be created from input of specific data into a device, for example a handheld device, suitably via an interface such as an app. Suitably the inputted data may then be utilised by the statistical models. Suitably a score may be graphically displayed.

Suitably “a high probability of acute heart failure” may be considered to mean an individual has an increased likelihood of having acute heart failure from a general population and individuals with no previous diagnosis of acute heart failure. Suitably a group considered at high probability of acute heart failure are those that would benefit from admission to hospital rather than discharge. Suitably those admitted to hospital may undergo suitable diagnostic tests and treatment. This treatment may be early life saving treatment. Suitably high probability of acute heart failure may be considered in terms of PPV and specificity. Suitably a PPV of 75% and specificity of 90% may be provided.

Suitably if the individual patient's probability score is above the high-probability threshold, then this is indicative that that the individual has a high probability of having acute heart failure.

Suitably if the individual patient's probability score is below the low-probability threshold, then this is indicative that that individual is at low probability of having acute heart failure.

Suitably, individual clinicians or healthcare institutions may select different optimal low- and high-probability score thresholds that correspond to the diagnostic performance that is most suited to the local setting.

Suitably any suitable assay method may be used to determine the level of natriuretic peptide, for example the level of NT-proBNP. For example, the assay method can be an immunoassay, for example an ELISA test. Suitably the assay may provide a level of a particular natriuretic peptide, for example a level of NT-proBNP.

Suitably, the step of obtaining values for least two other factors may comprise receiving values for a factor from an electronic individual's health record, receiving values inputted by a clinician based on a value obtained from the individual, receiving a value from a testing laboratory, or receiving a value from an electronic readout of a point of care device.

Suitably a sample from an individual may be a blood sample, suitably whole blood, serum, or plasma.

Suitably, the assay is based on the detection of one or more natriuretic peptides selected from the group consisting of atrial natriuretic peptide (“ANP”), proANP, NT-proANP, B-type natriuretic peptide (“BNP”), NT-pro BNP, pro-BNP, Mid-regional pro-atrial natriuretic peptide (MR-proANP) and C-type natriuretic peptide. In embodiments assays detect one or more natriuretic peptides selected from the group consisting of BNP, NT-pro BNP, and pro-BNP and in particular embodiments the detection and measurement of NT-proBNP. In embodiments assays detect one or more natriuretic peptides selected from the group consisting of BNP, NT-pro BNP and MR-proANP.

Detection can be by an assay that generates a detectable signal indicative of the presence or amount of a physiologically relevant concentration of that marker. Such an assay may, but need not, specifically detect a particular natriuretic peptide (e.g., detect BNP but not proBNP). If the assay detects an antibody epitope, then it would be understood by those of skill in the art, that if the epitope is on the order of 8 amino acids, the immunoassay will detect other polypeptides (e.g., related markers) so long as the other polypeptides contain the epitope(s) necessary to bind to the antibody used in the assay. As examples, NT-ProBNP can be measured on the Cobas (Roche Diagnostics) or the Atellica (Siemens Healthineers) platforms, BNP can be measured on the ARCHITECT platform (Abbott Diagnostics) and MR-proANP can be measured on the BRAHMS Kryptor platform (Thermo Fisher)

Suitably the method may comprise a treatment step. Suitably a treatment for an individual considered to be at high probability of acute heart failure may comprise, heart failure medications or performing additional diagnostic test or tests for example transthoracic echocardiography, ongoing monitoring of the individual in a critical care environment.

Embodiments of the present invention will now be described by way of example only, with reference to the accompanying figures in which:

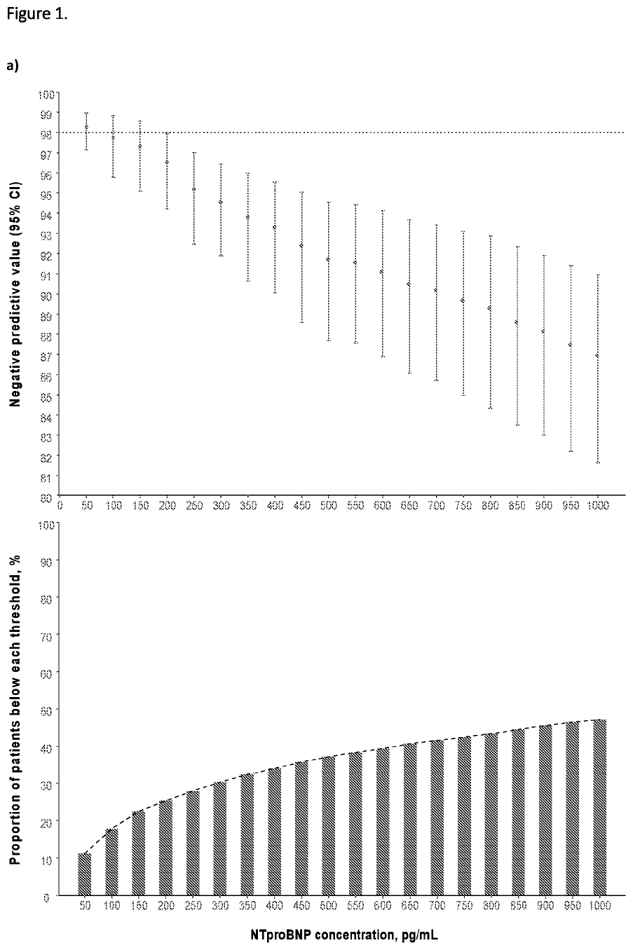

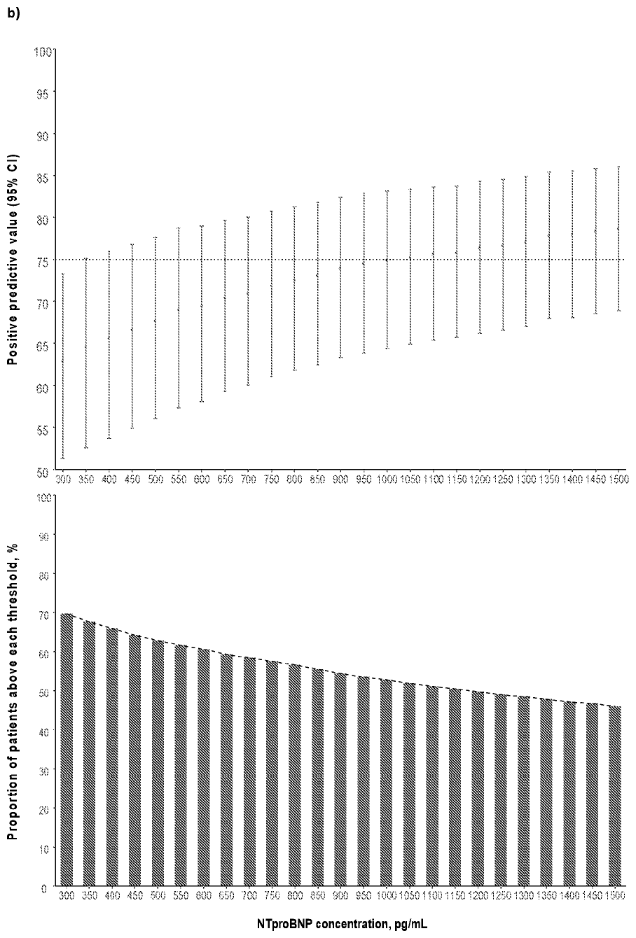

FIG. 1 illustrates NT-proBNP thresholds for acute heart failure (a) (top) where Negative predictive values of NT-proBNP concentrations to rule-out a diagnosis of acute heart failure. (bottom) Cumulative proportion of patients presenting with suspected acute heart failure with NT-proBNP concentrations below each threshold, (b) (top) Positive predictive values of NT-proBNP concentrations to rule-in a diagnosis of acute heart failure. (bottom) Cumulative proportion of patients presenting with suspected acute heart failure with NT-proBNP concentrations above each threshold.

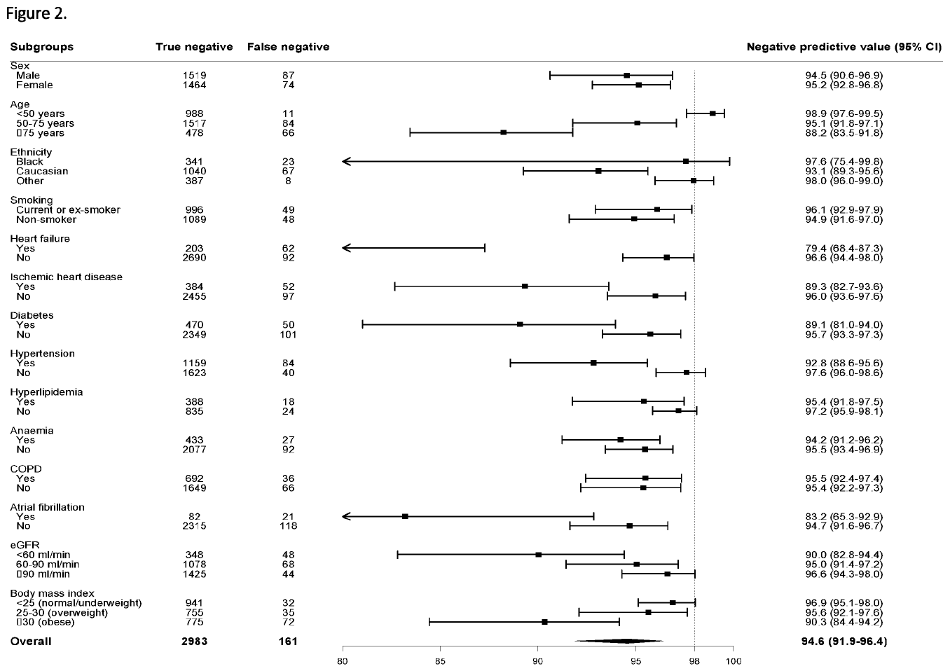

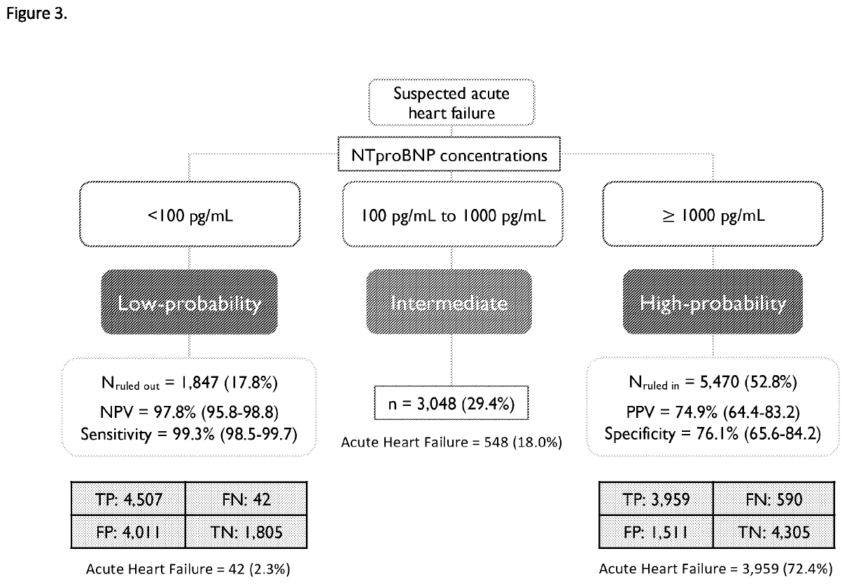

FIG. 2 illustrates Negative predictive value of the NT-proBNP threshold of 300 pg/mL across patient subgroups where pooled meta-estimates of negative predictive value within prespecified patient subgroups were derived using random-effects meta-analysis. Abbreviations: COPD=chronic obstructive pulmonary disease; eGFR=estimated glomerular filtration rate FIG. 3 illustrates a diagnostic pathway for acute heart failure using optimized NT-proBNP thresholds wherein proposed diagnostic pathway for acute heart failure uses NT-proBNP thresholds that meet target rule-in and rule-out criteria of 75% PPV and 98% NPV, respectively. Abbreviations: TP=true positive, FP=false positive, TN=true negative, FN=false negative.

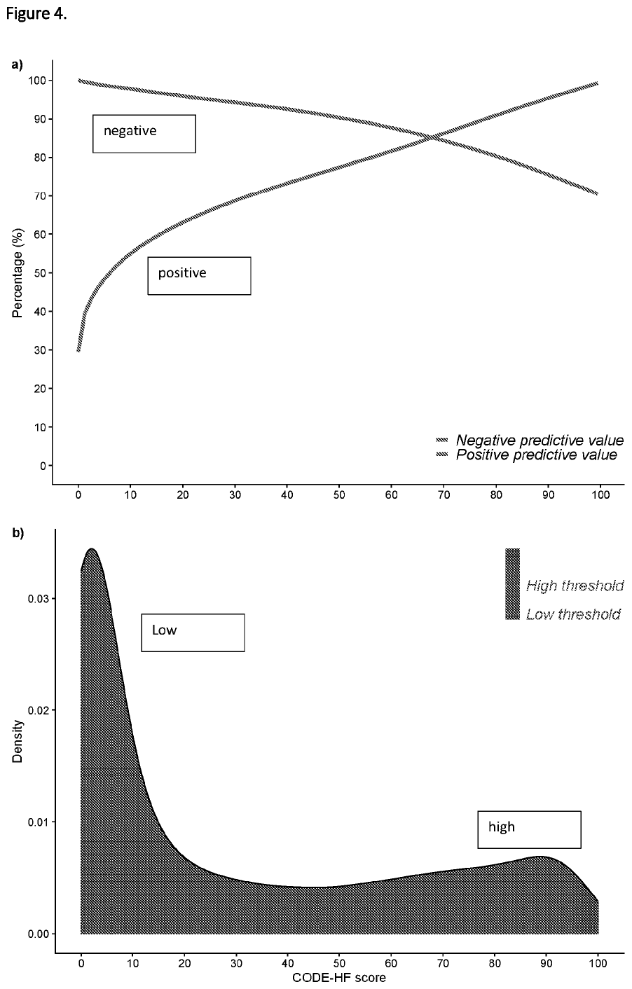

FIG. 4 illustrates diagnostic performance of the CoDE-HF score in patients without prior heart failure wherein (a) Negative and positive predictive values of CoDE-HF scores. Blue vertical dashed line=target rule-out score of 5.7. Red vertical dashed line=target rule-in score of 45.2, (b) Density plot of CoDE-HF score in patients without prior heart failure. The target rule-out and rule-in scores identify 42.3% of patients as low-probability and 30.5% as high-probability respectively based on the GLMM and XGBoost models generated using the approach taught therein.

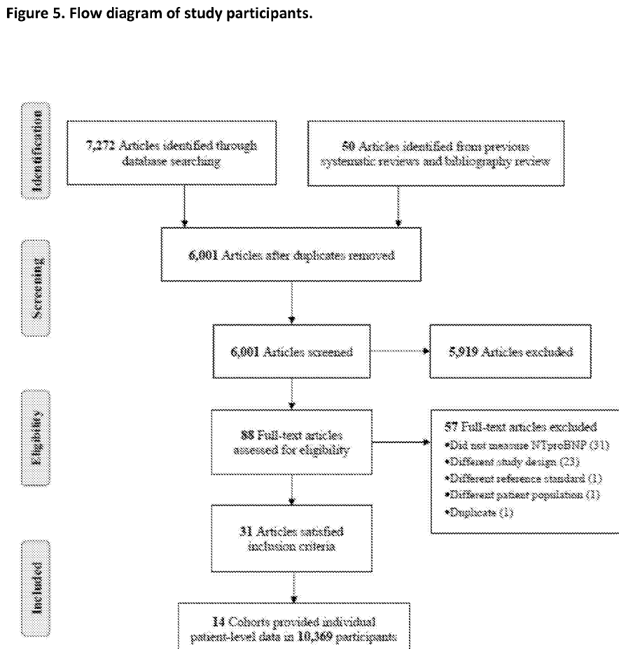

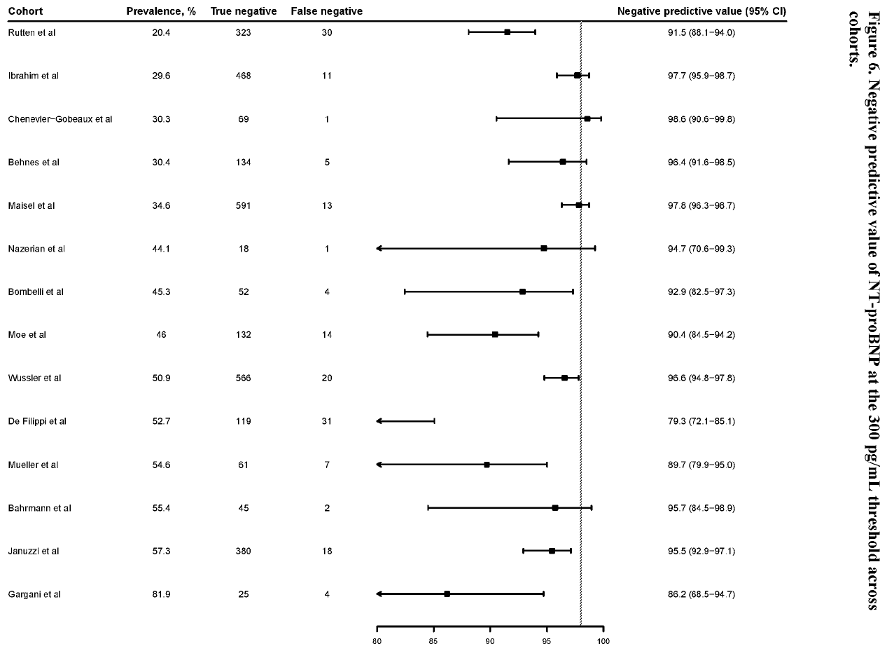

FIG. 5 illustrates a flow diagram of study participants.

FIG. 6 illustrates a negative predictive value of NT-proBNP at the 300 pg/mL threshold across cohorts.

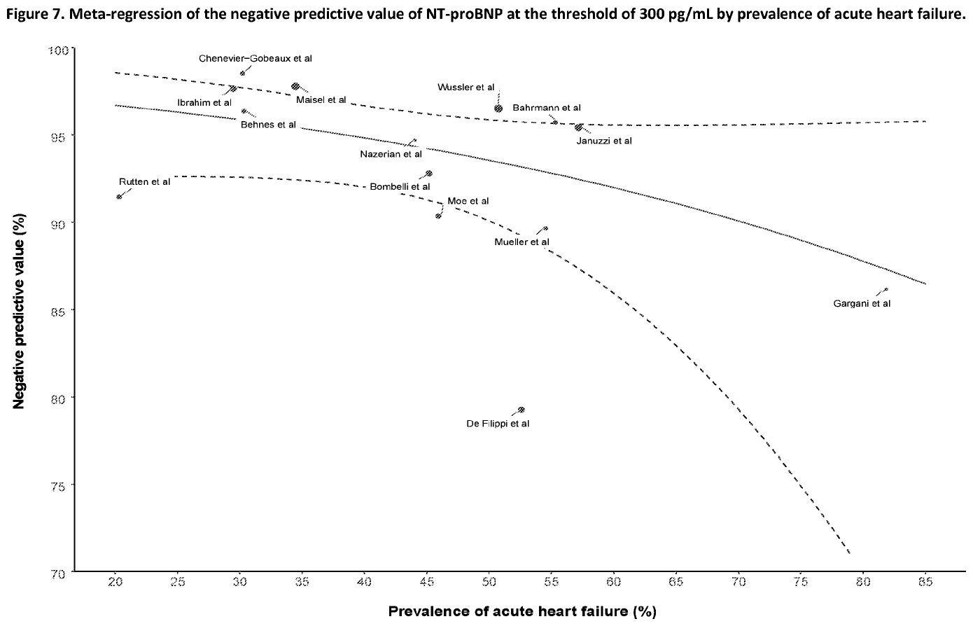

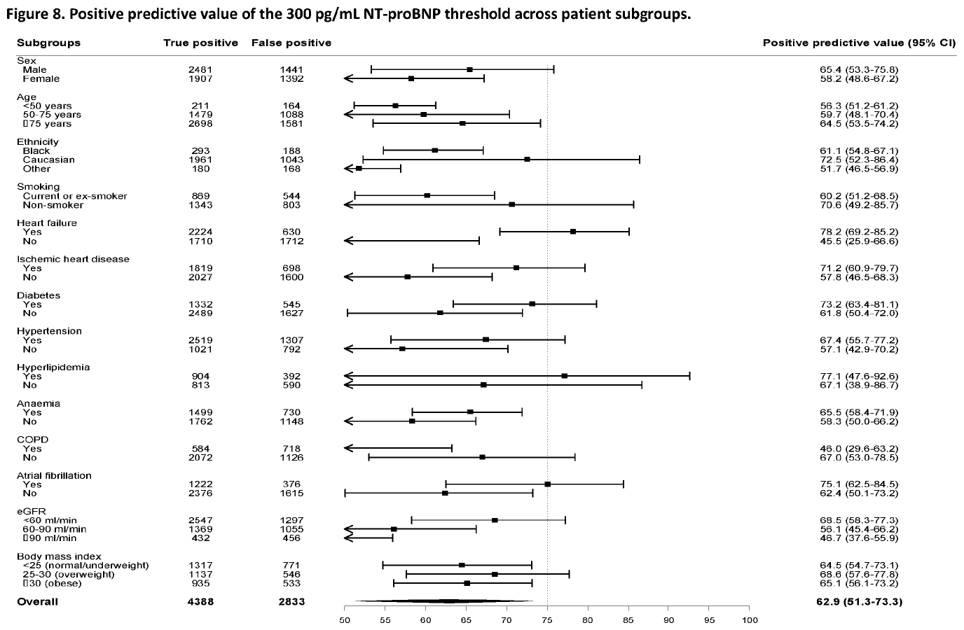

FIG. 7 illustrates a meta-regression of the negative predictive value of NT-proBNP at the threshold of 300 pg/mL by prevalence of acute heart failure FIG. 8 illustrates a positive predictive value of the 300 pg/mL NT-proBNP threshold across patient subgroups

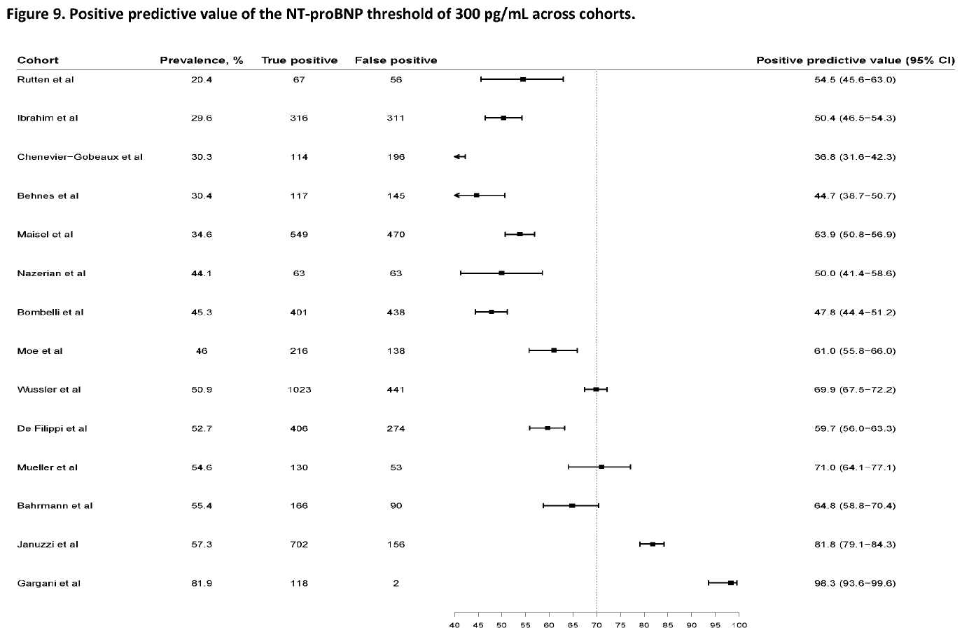

FIG. 9 illustrates a positive predictive value of the NT-proBNP threshold of 300 pg/mL across cohorts.

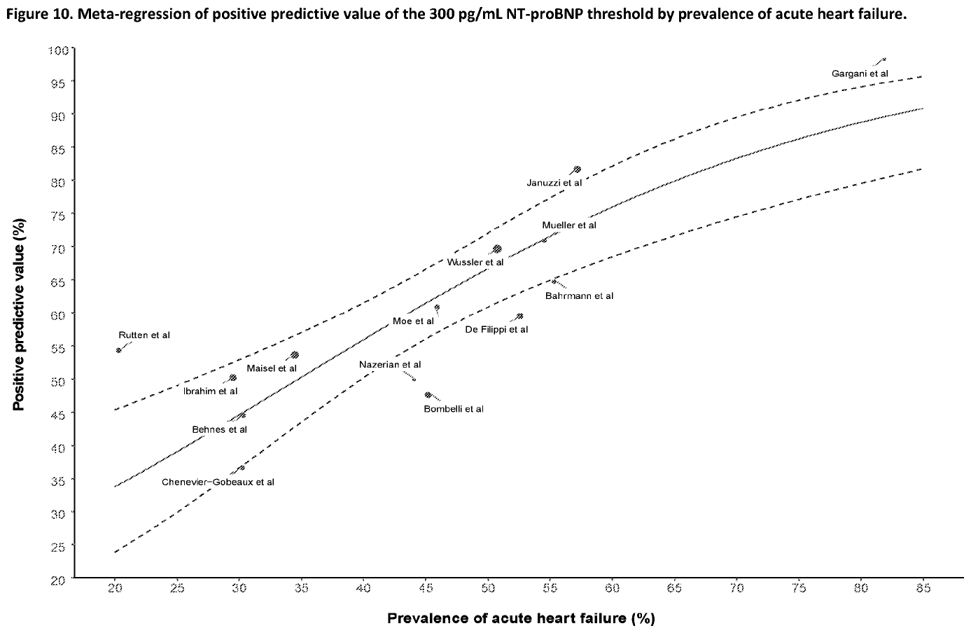

FIG. 10 illustrates a meta-regression of positive predictive value of the 300 pg/mL NT-proBNP threshold by prevalence of acute heart failure.

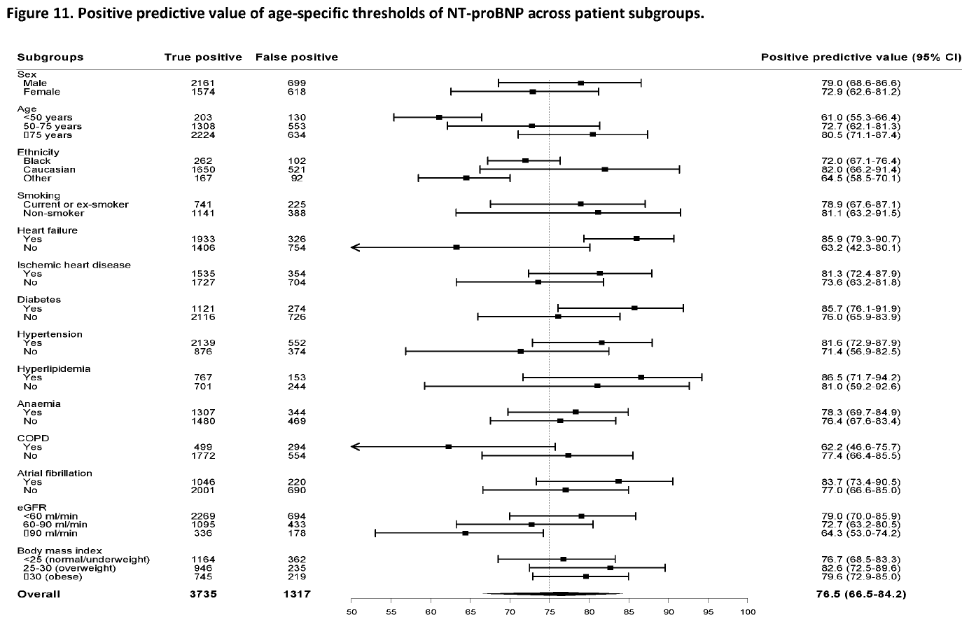

FIG. 11 illustrates a positive predictive value of age-specific thresholds of NT-proBNP across patient subgroups.

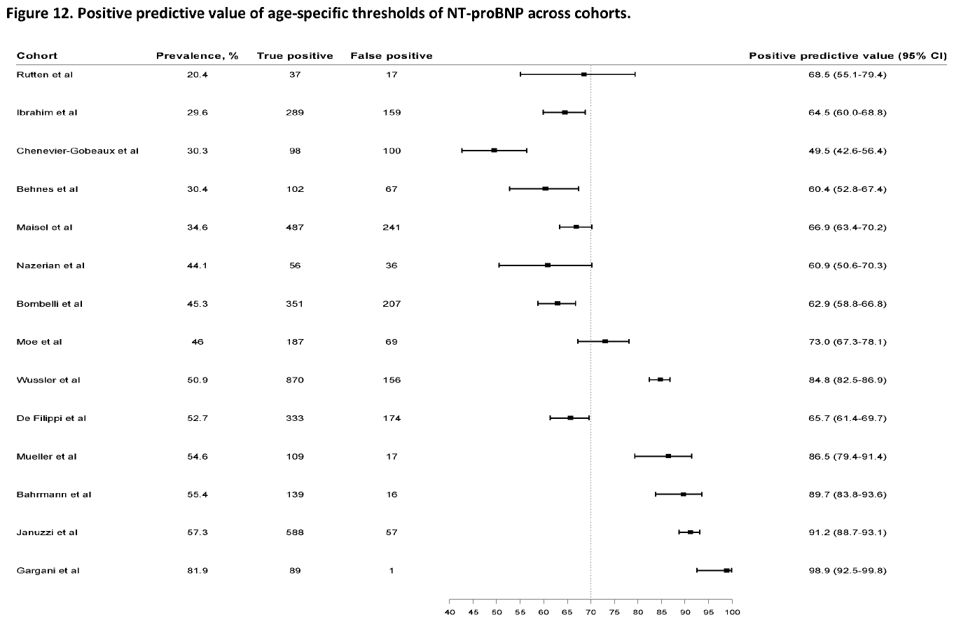

FIG. 12 illustrates a positive predictive value of age-specific thresholds of NT-proBNP across cohorts.

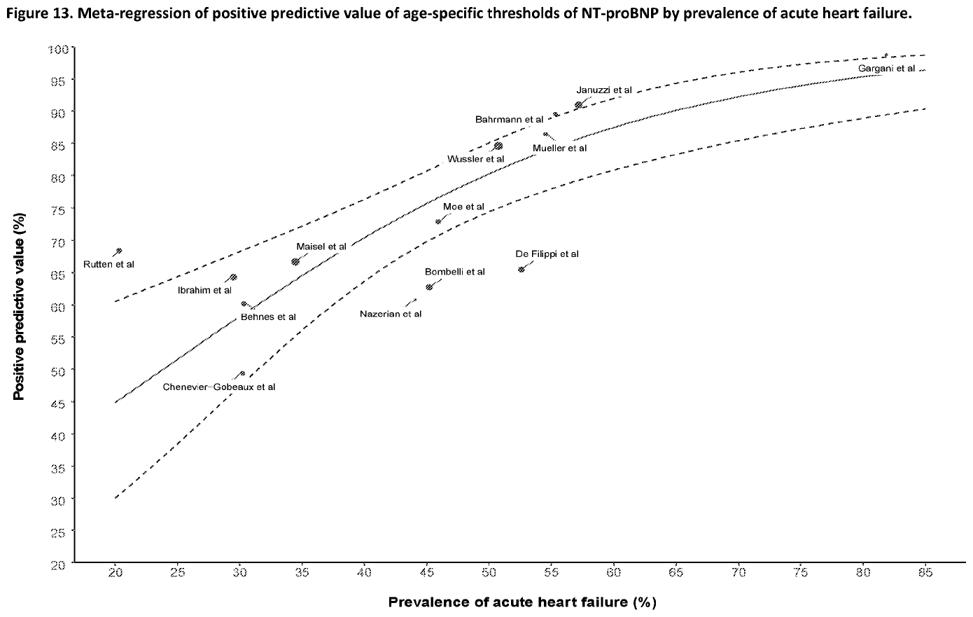

FIG. 13 illustrates meta-regression of positive predictive value of age-specific thresholds of NT-proBNP by prevalence of acute heart failure.

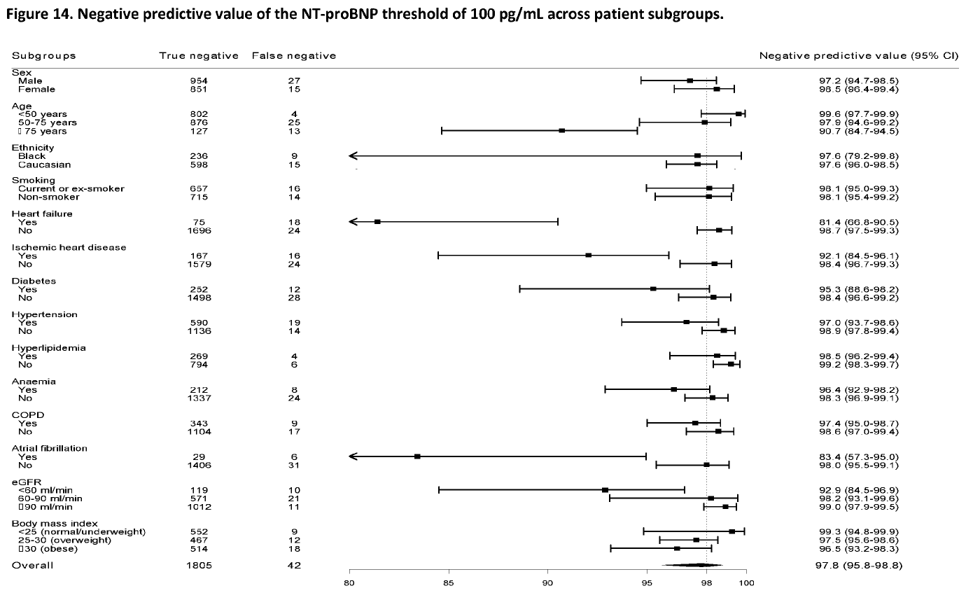

FIG. 14 illustrates a negative predictive value of the NT-proBNP threshold of 100 pg/mL across patient subgroups.

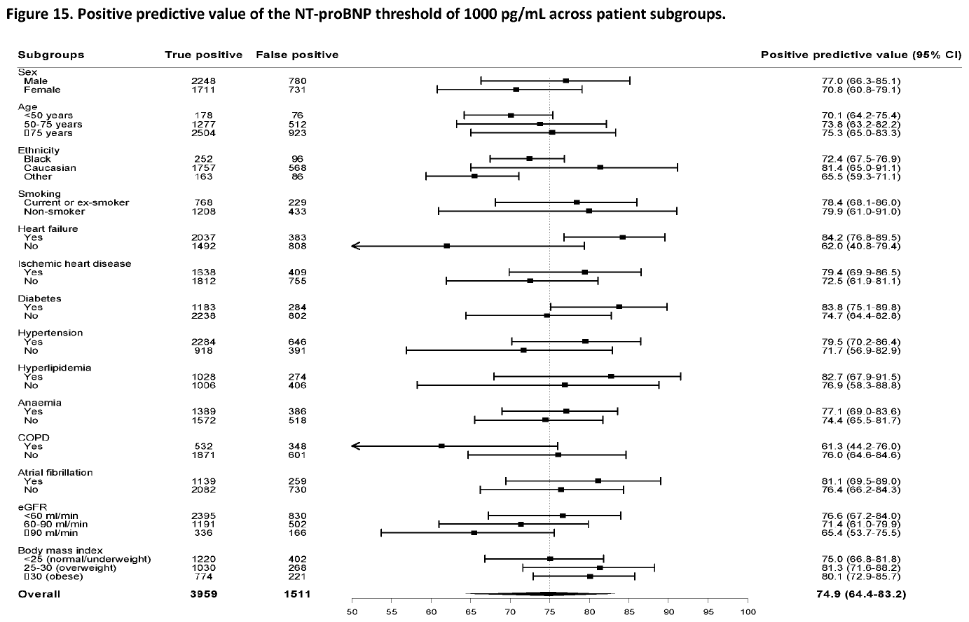

FIG. 15 illustrates a positive predictive value of the NT-proBNP threshold of 1000 pg/mL across patient subgroups.

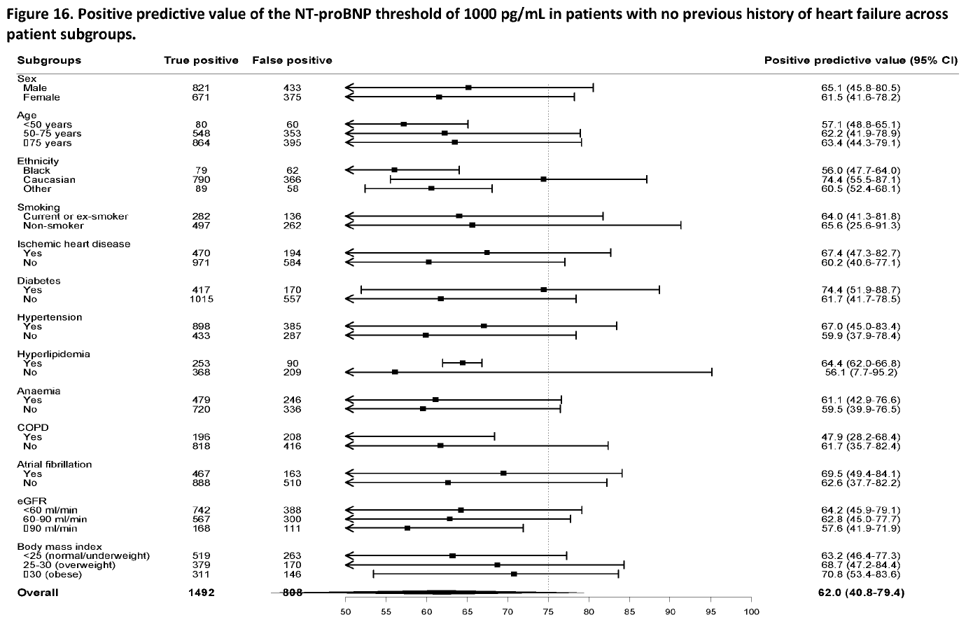

FIG. 16 illustrates a positive predictive value of the NT-proBNP threshold of 1000 pg/mL in patients with no previous history of heart failure across patient subgroups.

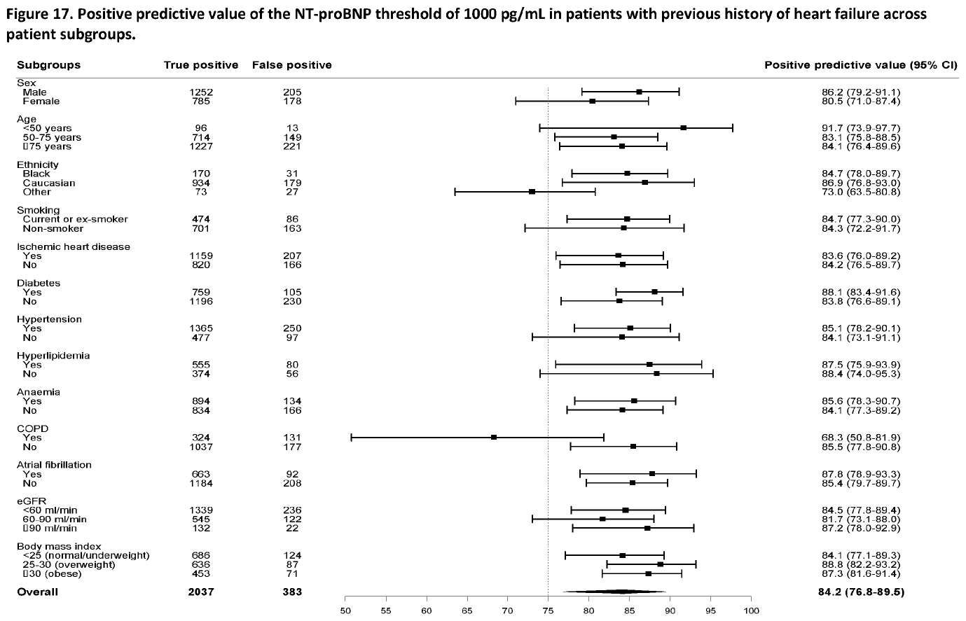

FIG. 17 illustrates a positive predictive value of the NT-proBNP threshold of 1000 pg/mL in patients with previous history of heart failure across patient subgroups.

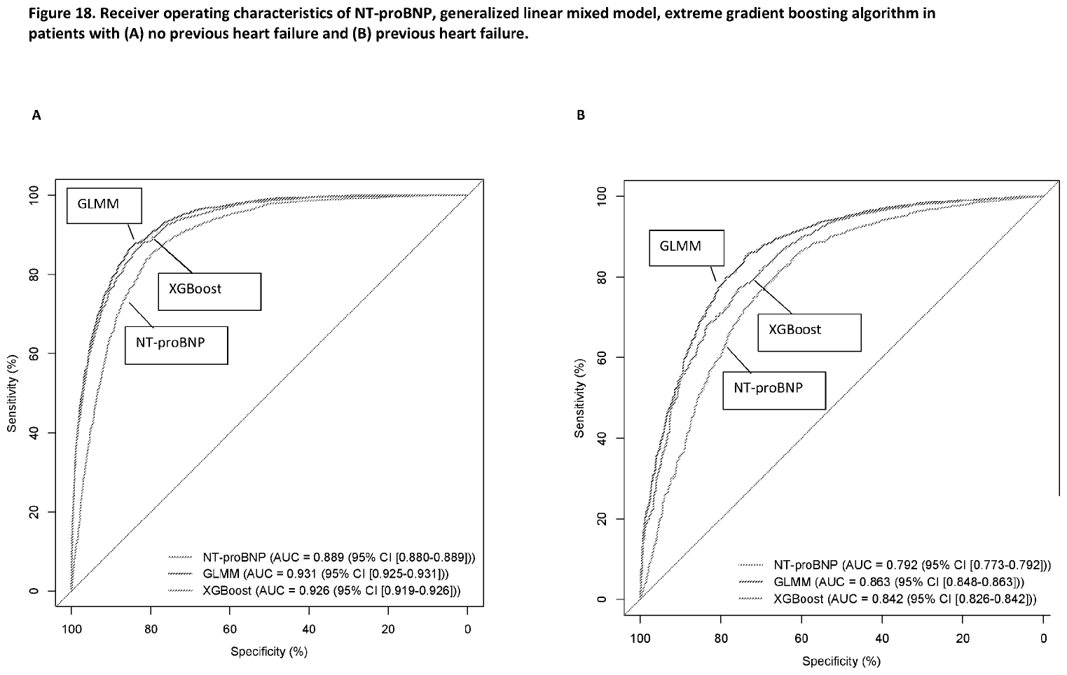

FIG. 18 illustrates a receiver operating characteristics of NT-proBNP, generalized linear mixed model, extreme gradient boosting algorithm in patients with (A) no previous heart failure and (B) previous heart failure.

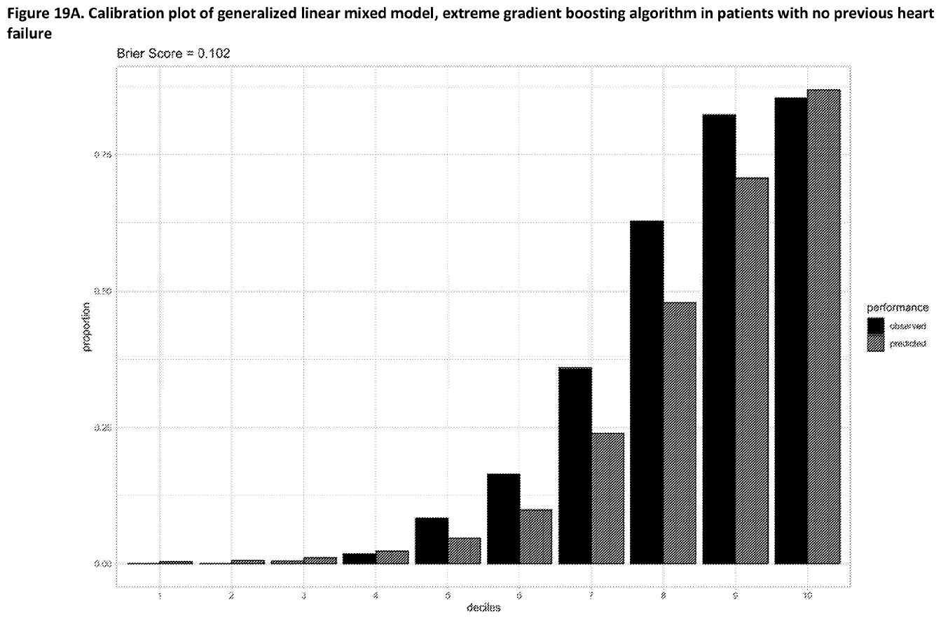

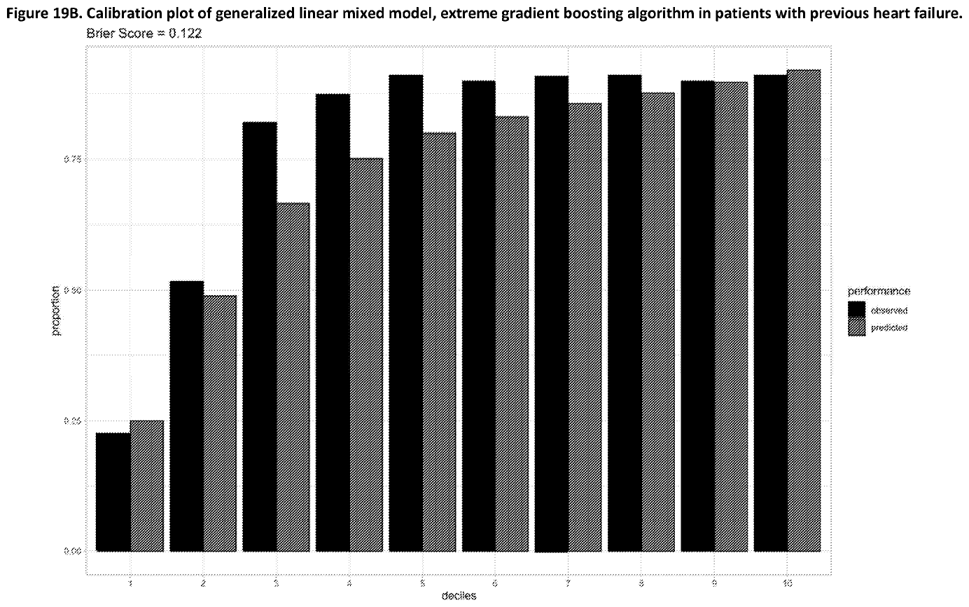

FIG. 19 illustrates a calibration plot of generalized linear mixed model, extreme gradient boosting algorithm in patients with (A) no previous heart failure and (B) previous heart failure.

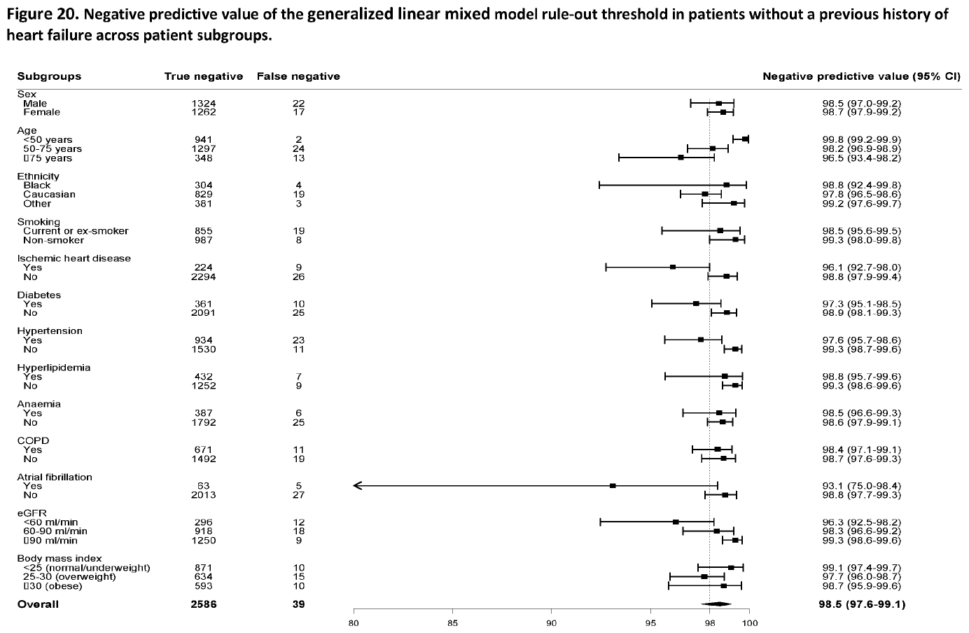

FIG. 20 illustrates a negative predictive value of the generalized linear mixed model rule-out threshold in patients without a previous history of heart failure across patient subgroups.

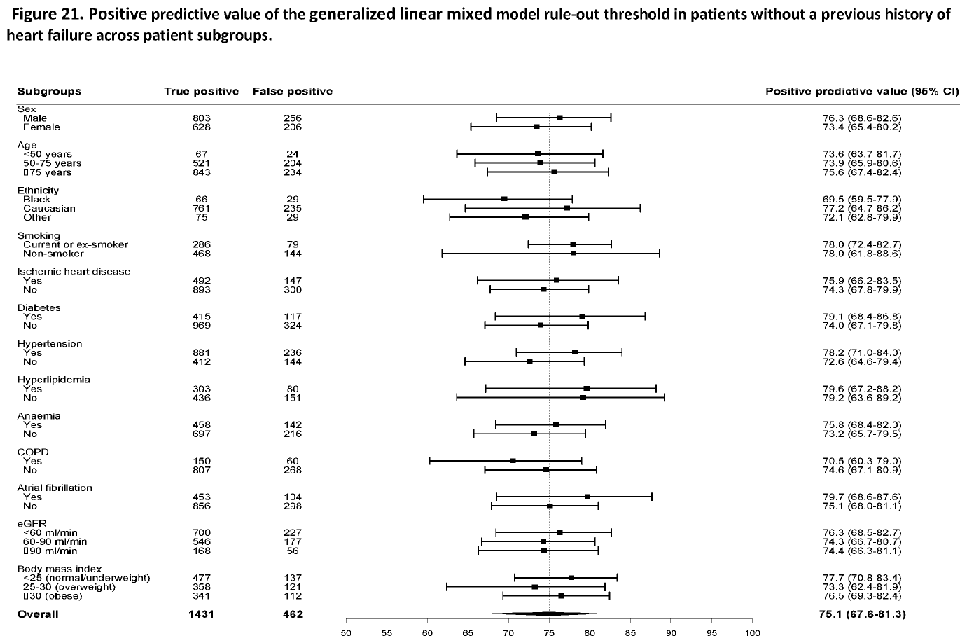

FIG. 21 illustrates a positive predictive value of the generalized linear mixed model rule-out threshold in patients without a previous history of heart failure across patient subgroups.

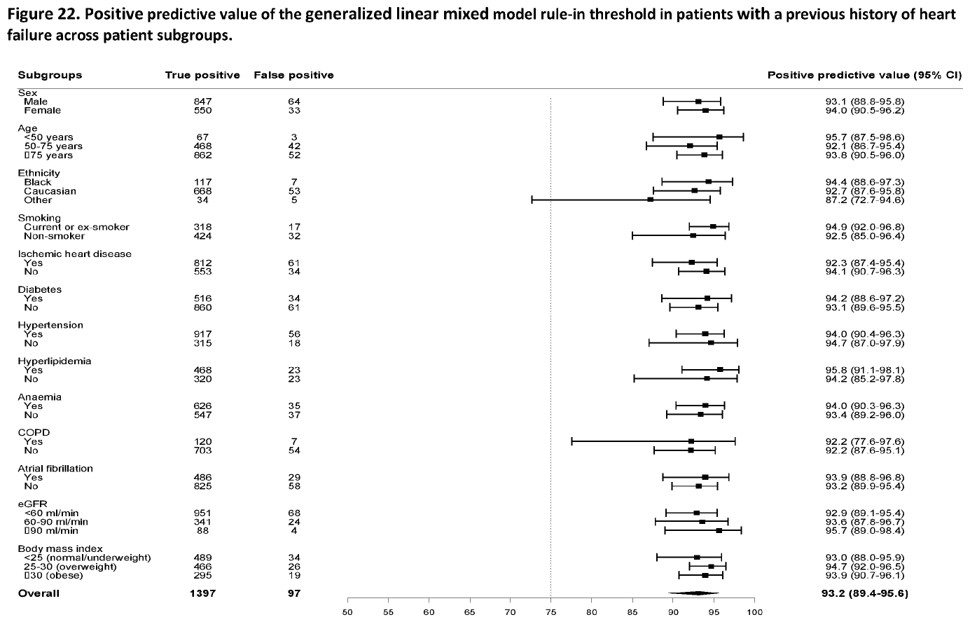

FIG. 22 illustrates a positive predictive value of the generalized linear mixed model rule-in threshold in patients with a previous history of heart failure across patient subgroups.

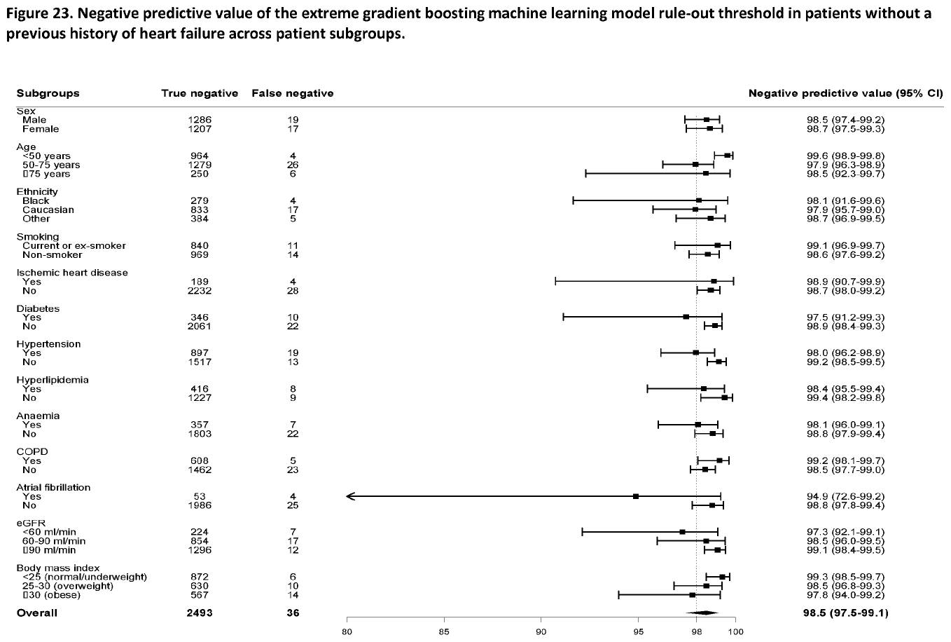

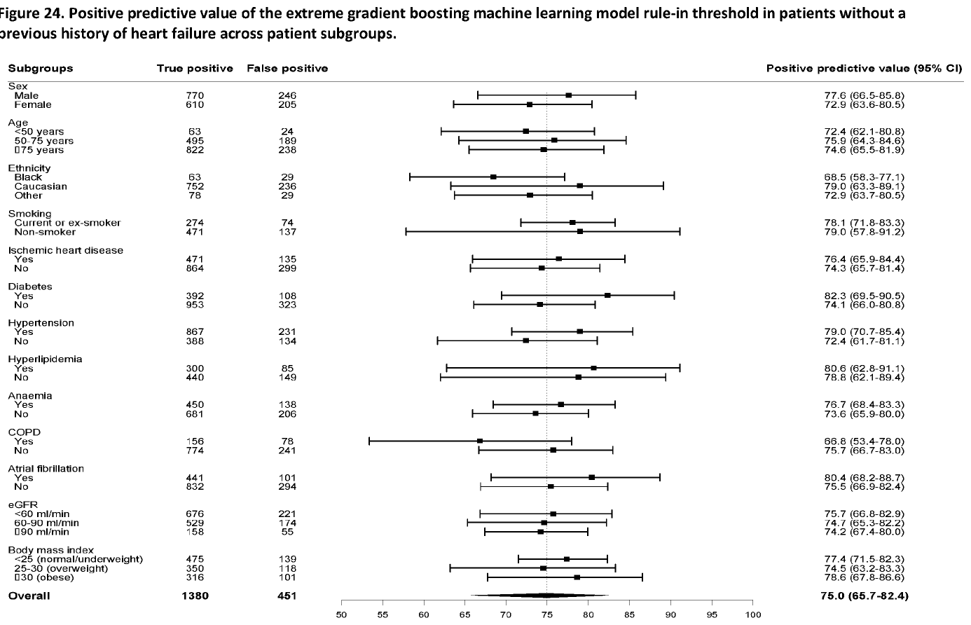

FIG. 23 illustrates a negative predictive value of the extreme gradient boosting machine learning model rule-out threshold in patients without a previous history of heart failure across patient subgroups.

FIG. 24 illustrates a positive predictive value of the extreme gradient boosting machine learning model rule-in threshold in patients without a previous history of heart failure across patient subgroups.

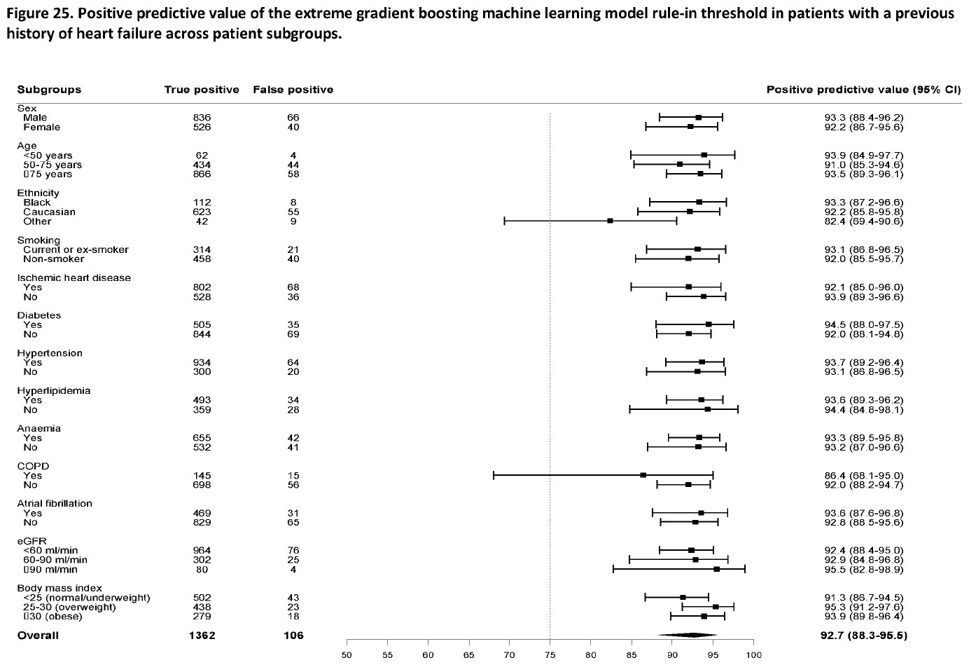

FIG. 25 illustrates a positive predictive value of the extreme gradient boosting machine learning model rule-in threshold in patients with a previous history of heart failure across patient subgroups.

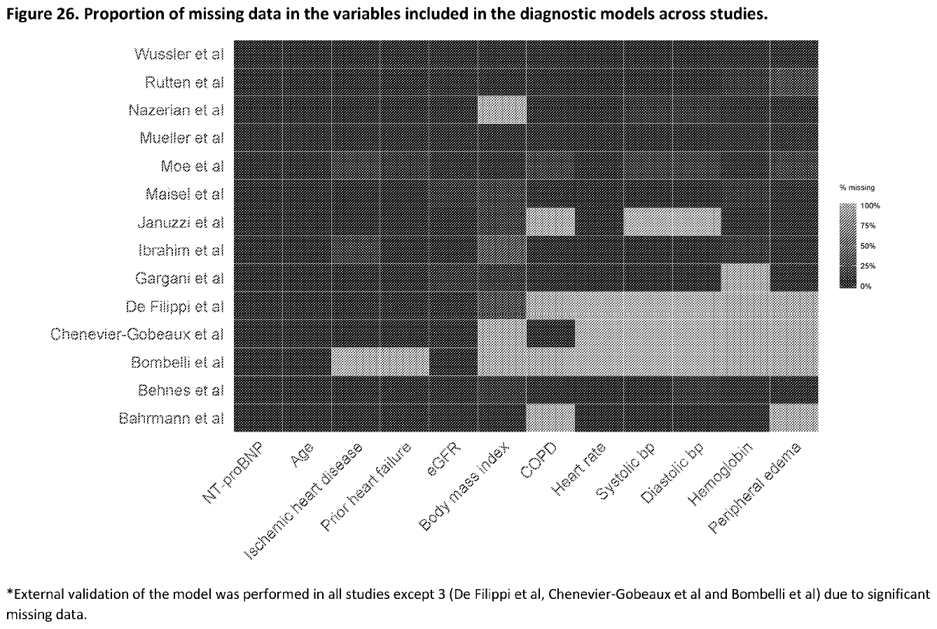

FIG. 26 illustrates a proportion of missing data in the variables included in the diagnostic models across studies.

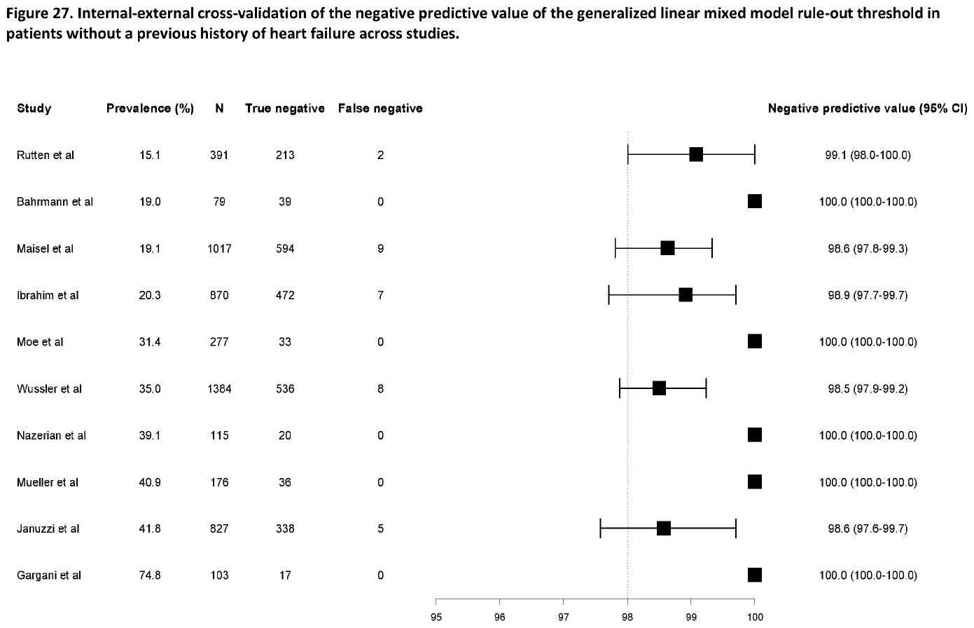

FIG. 27 illustrates a an internal-external cross-validation of the negative predictive value of the generalized linear mixed model rule-out threshold in patients without a previous history of heart failure across studies.

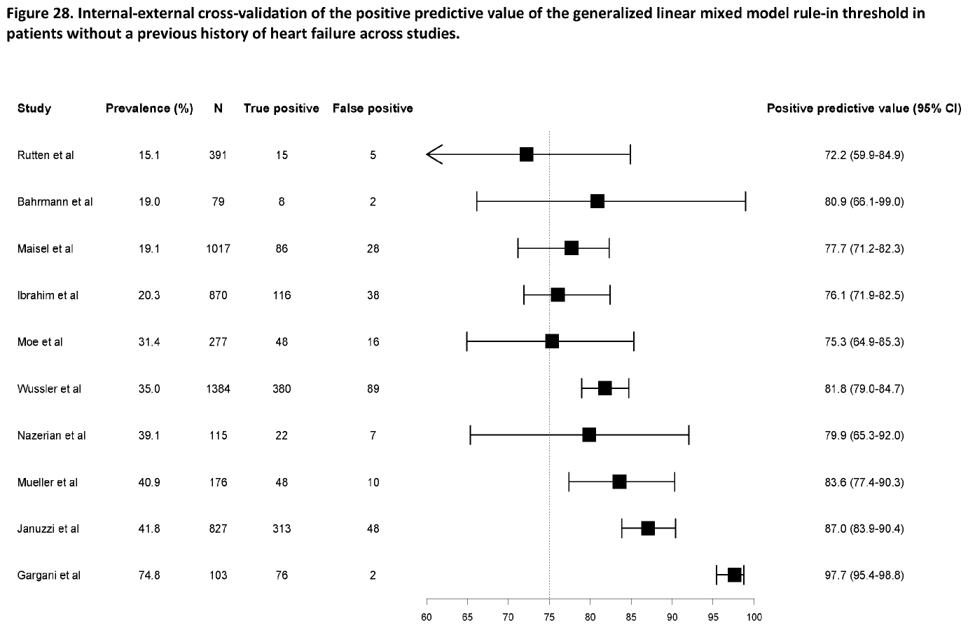

FIG. 28 illustrates an internal-external cross-validation of the positive predictive value of the generalized linear mixed model rule-in threshold in patients without a previous history of heart failure across studies.

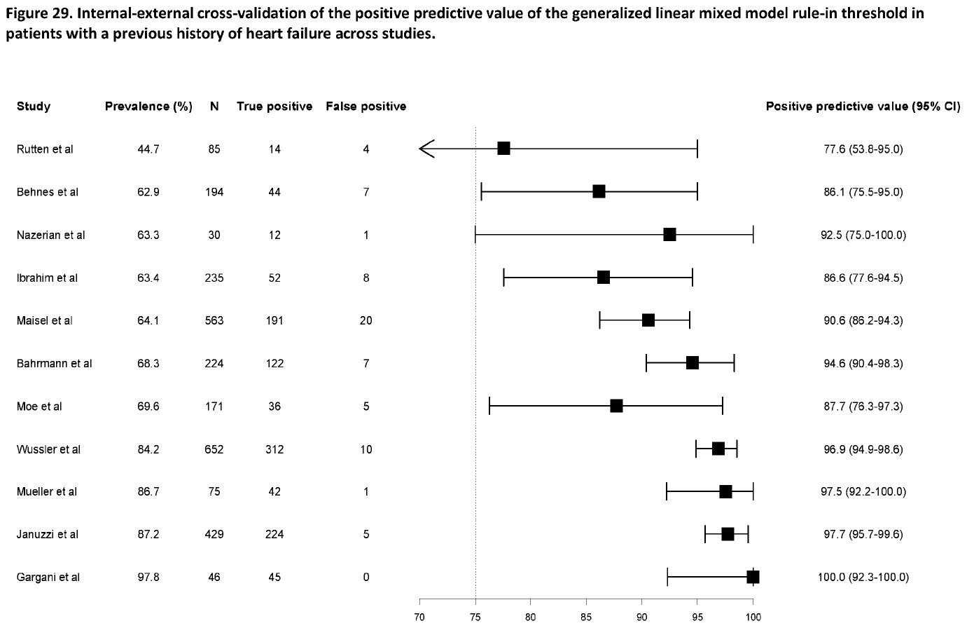

FIG. 29 illustrates an internal-external cross-validation of the positive predictive value of the generalized linear mixed model rule-in threshold in patients with a previous history of heart failure across studies.

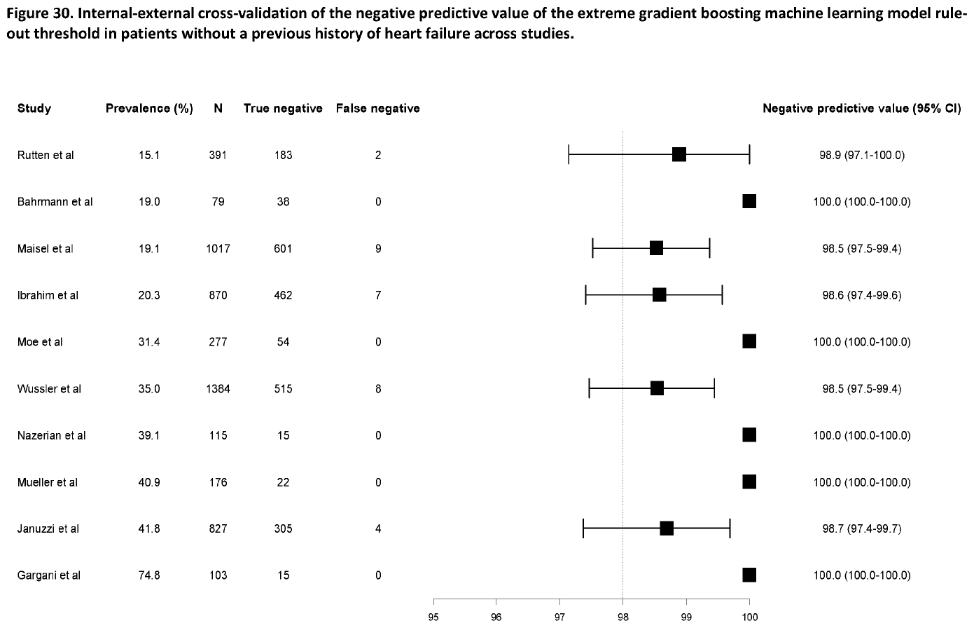

FIG. 30 illustrates an internal-external cross-validation of the negative predictive value of the extreme gradient boosting machine learning model rule-out threshold in patients without a previous history of heart failure across studies.

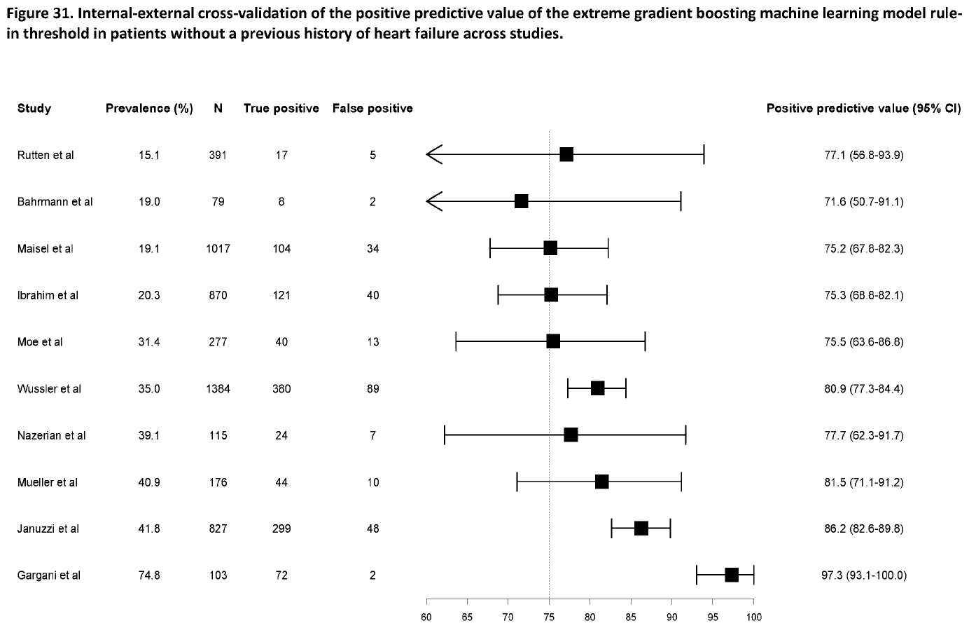

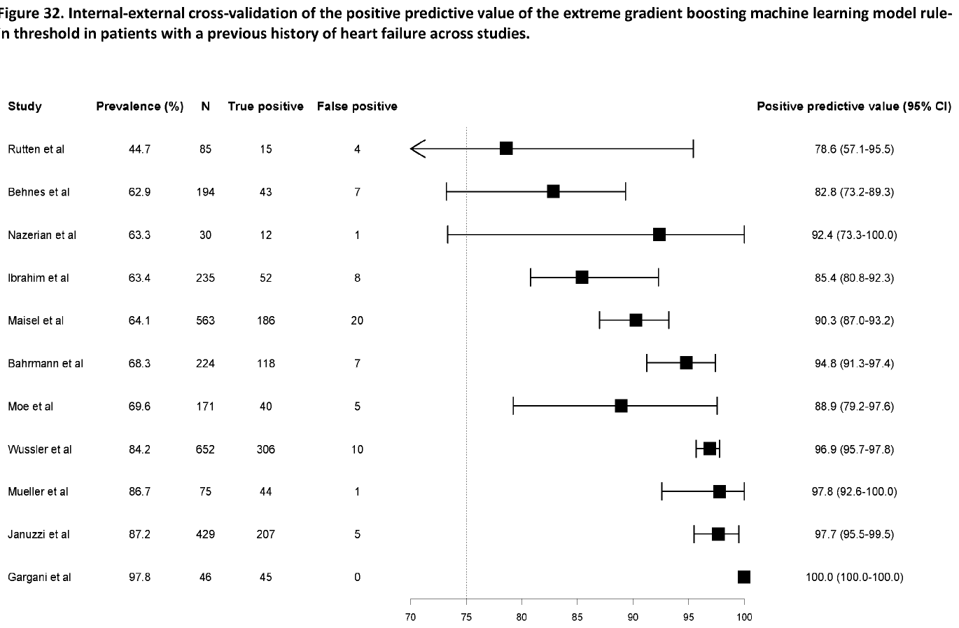

FIG. 31 illustrates an internal-external cross-validation of the positive predictive value of the extreme gradient boosting machine learning model rule-in threshold in patients without a previous history of heart failure across studies.

FIG. 32 illustrates an internal-external cross-validation of the positive predictive value of the extreme gradient boosting machine learning model rule-in threshold in patients with a previous history of heart failure across studies.

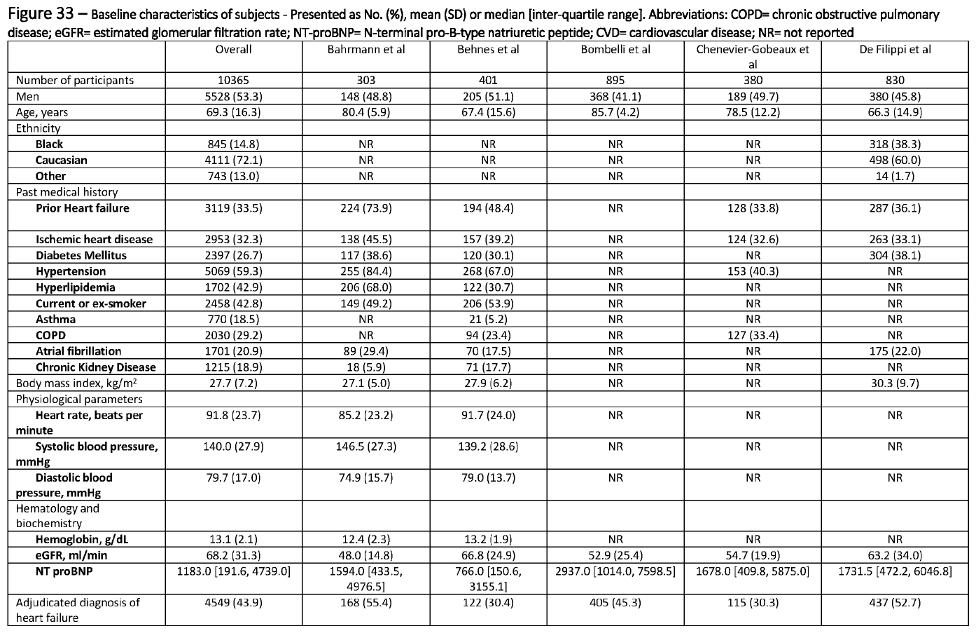

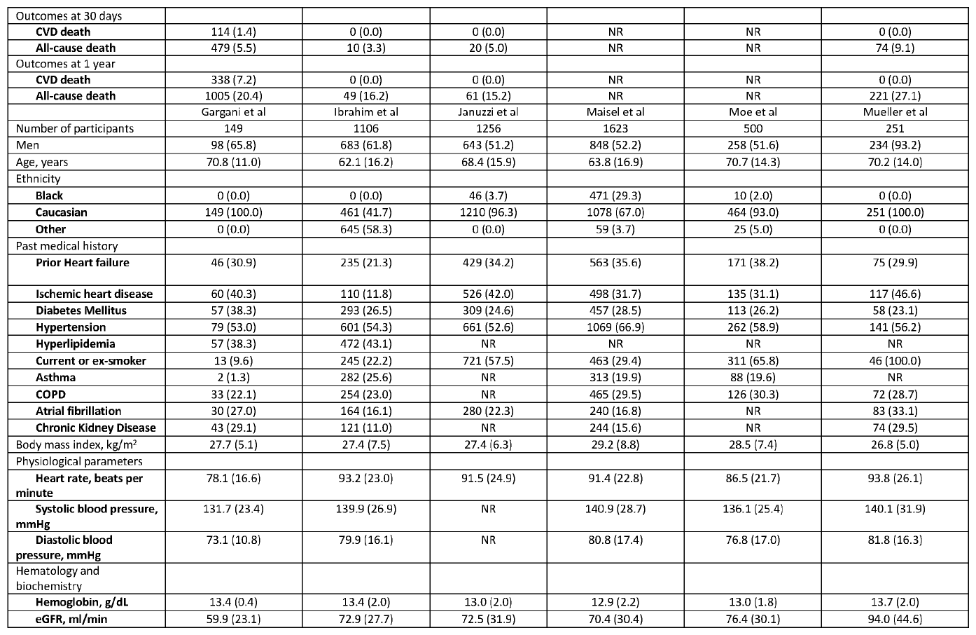

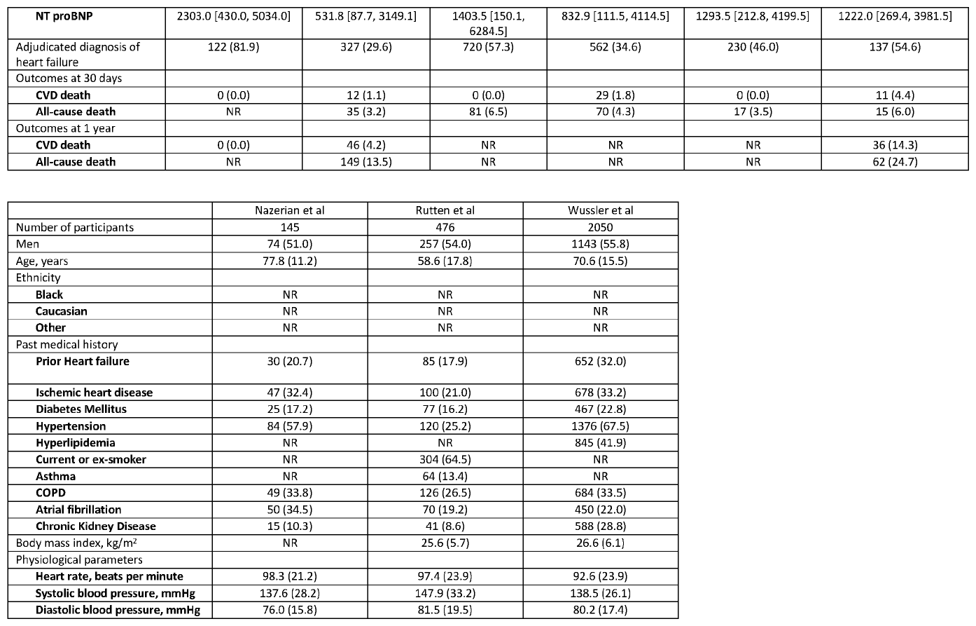

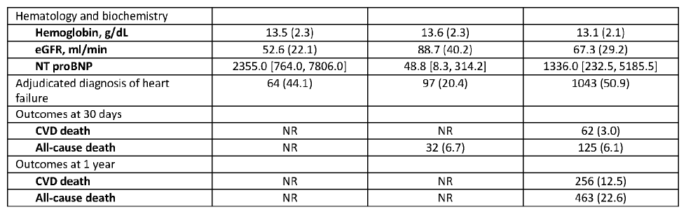

FIG. 33 illustrates baseline characteristics of subjects with each study —Presented as No. (%), mean (SD) or median [inter-quartile range]. Abbreviations: COPD=chronic obstructive pulmonary disease; eGFR=estimated glomerular filtration rate; NT-proBNP=N-terminal pro-B-type natriuretic peptide; CVD=cardiovascular disease; NR=not reported.

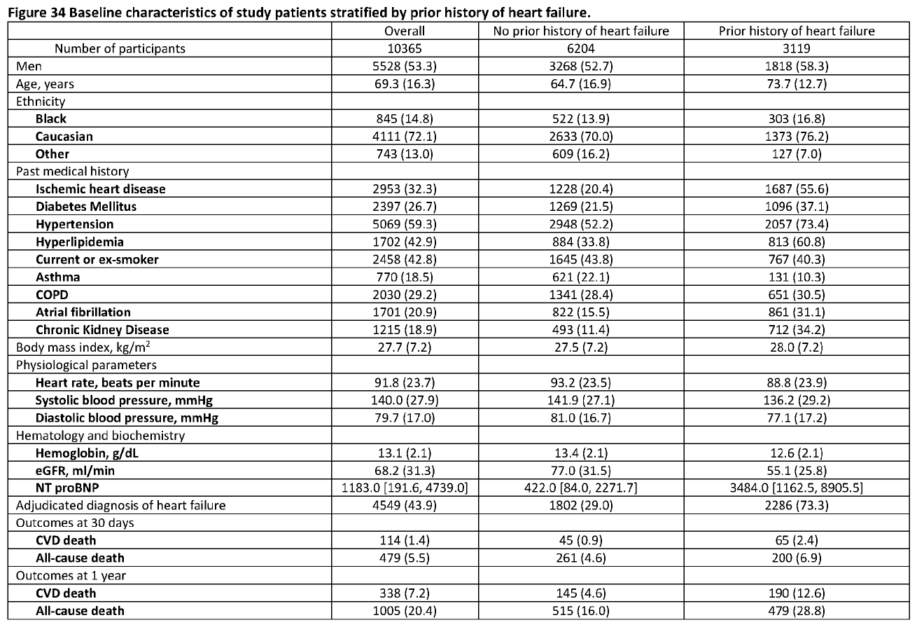

FIG. 34 illustrates baseline characteristics of study patients stratified by prior history of heart failure.

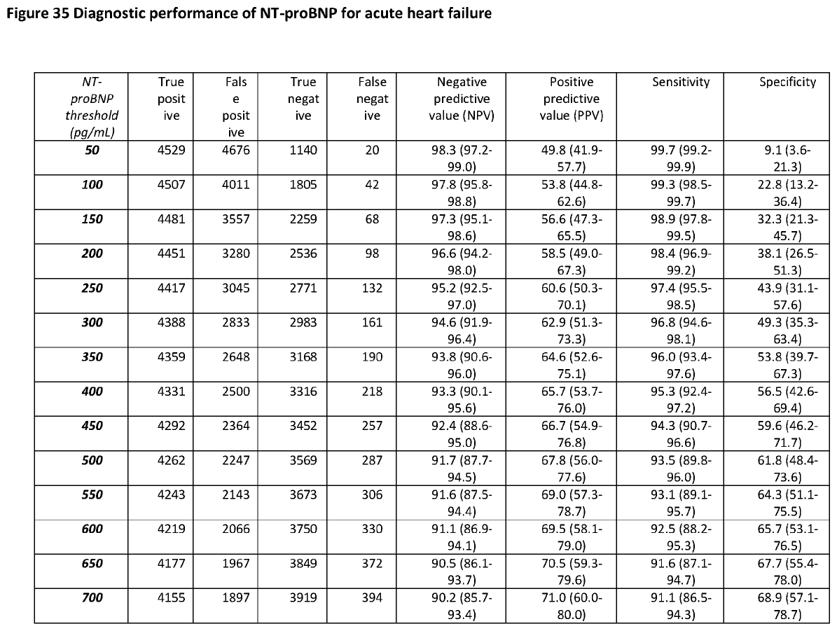

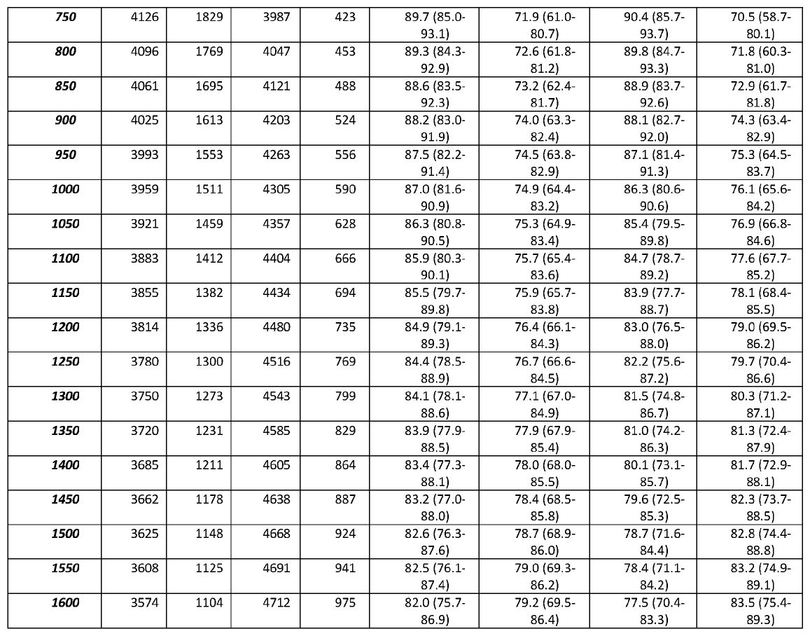

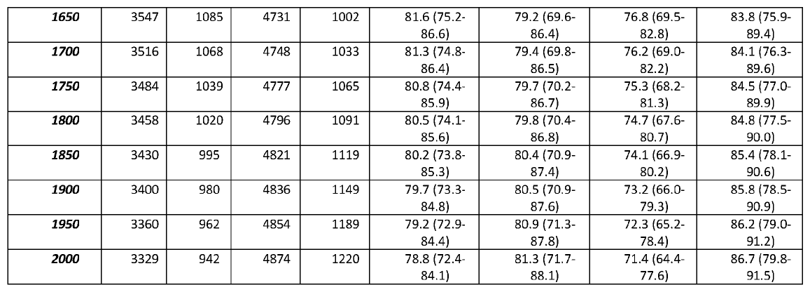

FIG. 35 illustrates diagnostic performance of NT-proBNP for acute heart failure.

FIG. 36 illustrates diagnostic performance of age-specific thresholds of NT-proBNP for acute heart failure.

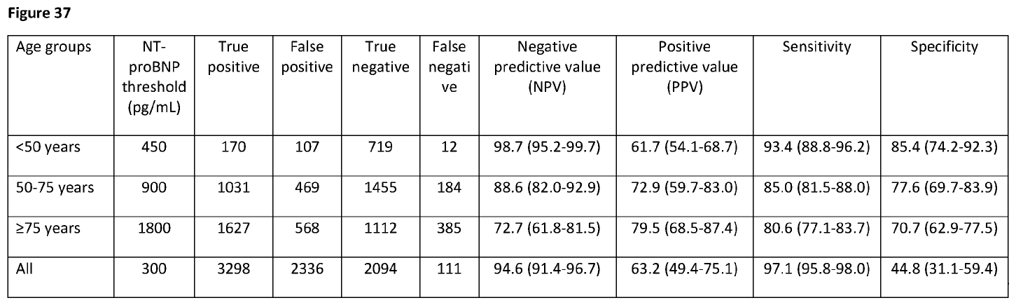

FIG. 37 illustrates diagnostic performance of age-specific thresholds of NT-proBNP for acute heart failure. Sensitivity analysis in studies where the reference standard was blinded to NT-proBNP concentration.

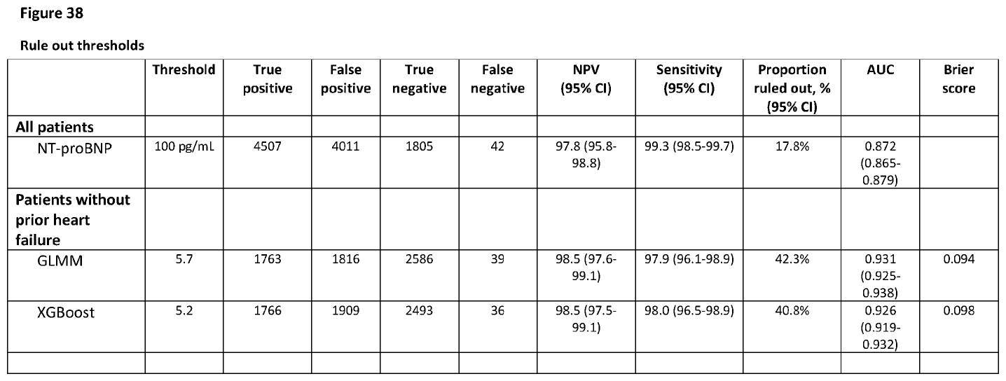

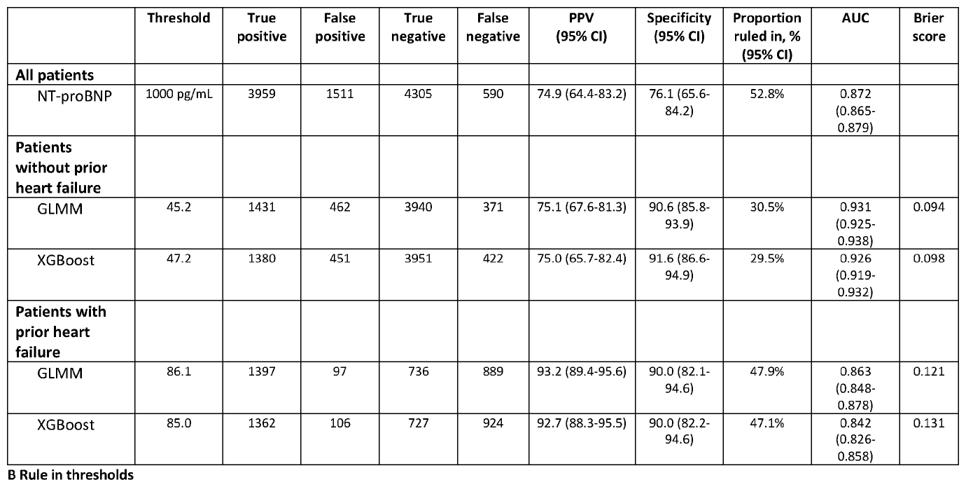

FIG. 38 illustrates (A) rule out thresholds (B) rule out thresholds.

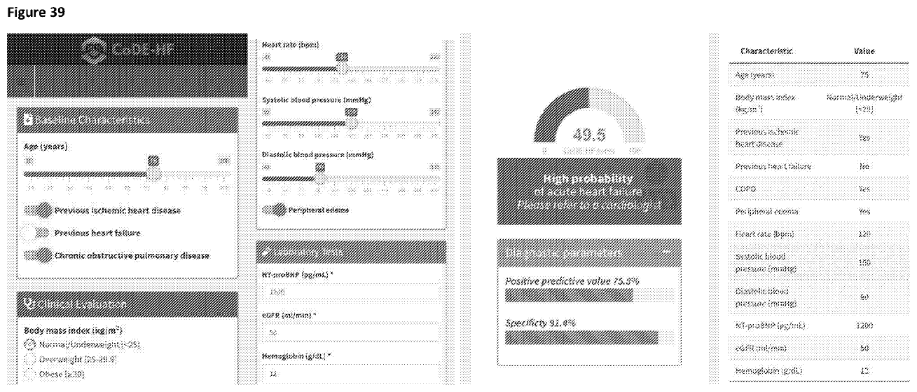

FIG. 39 illustrates input data into a system to determine a probability of Acute Heart disease.

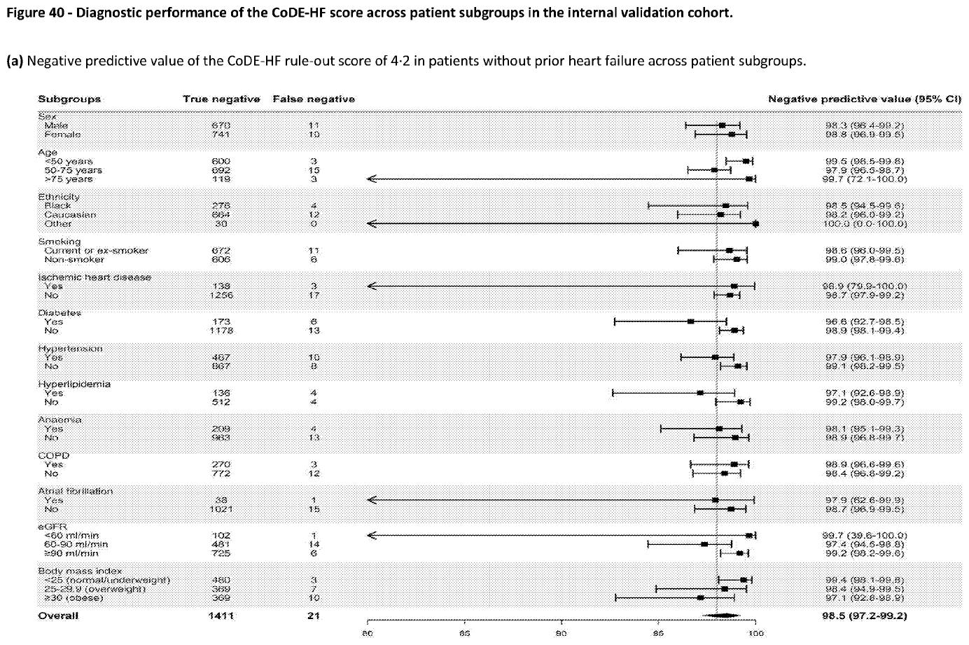

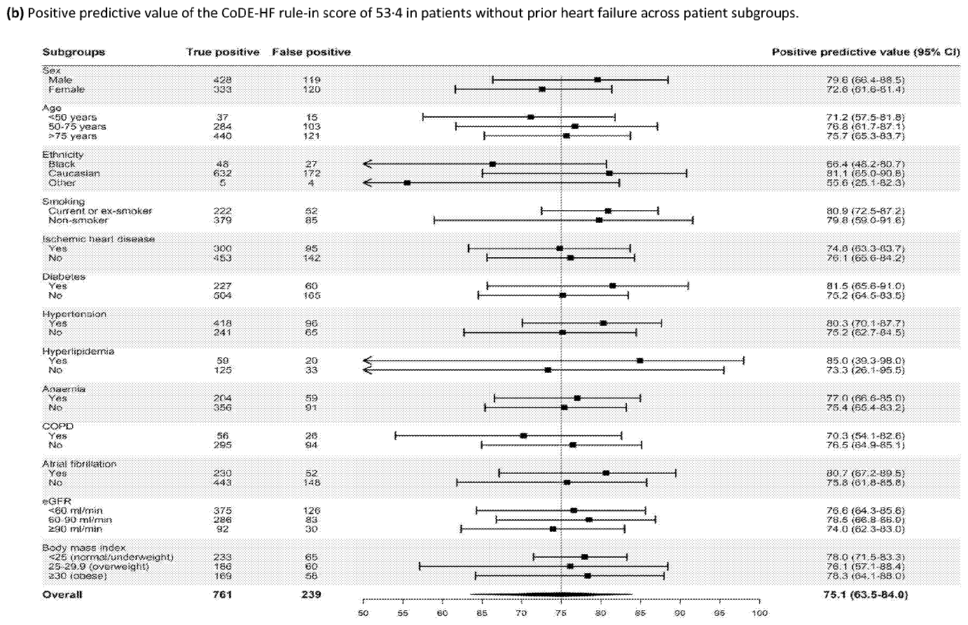

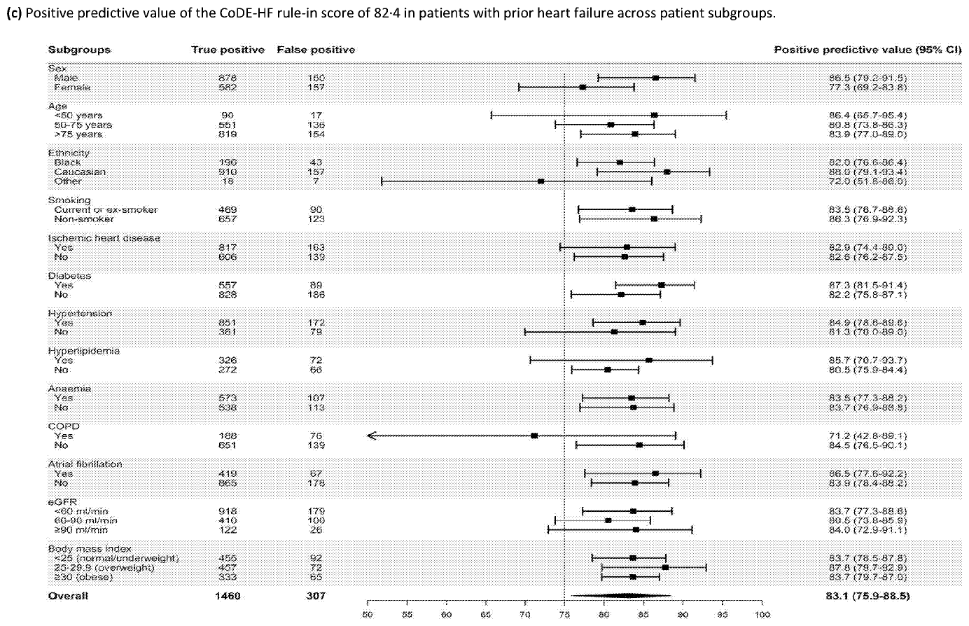

FIG. 40 illustrates Diagnostic performance of the CoDE-HF score across patient subgroups in the internal validation cohort.

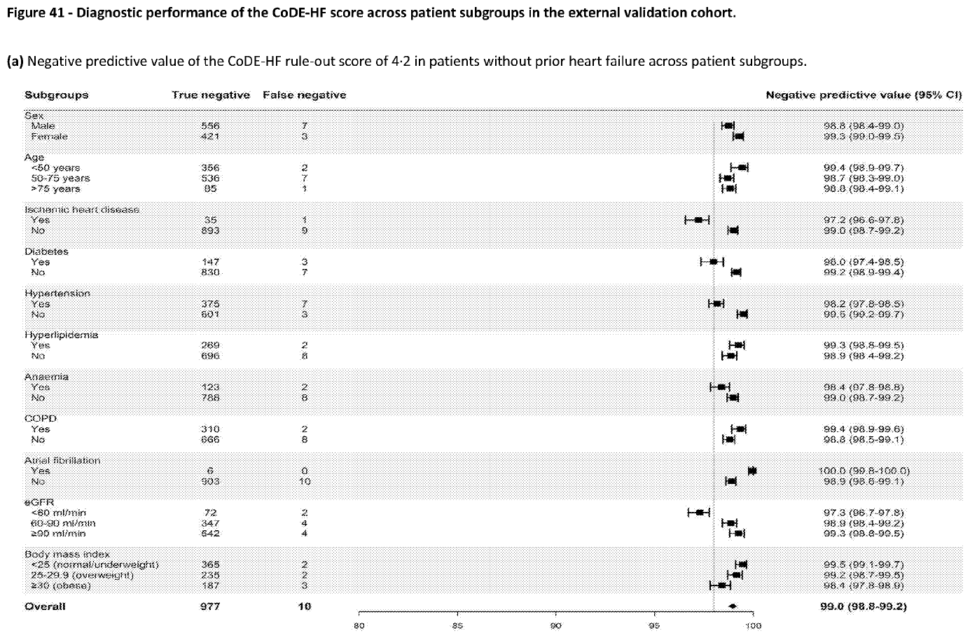

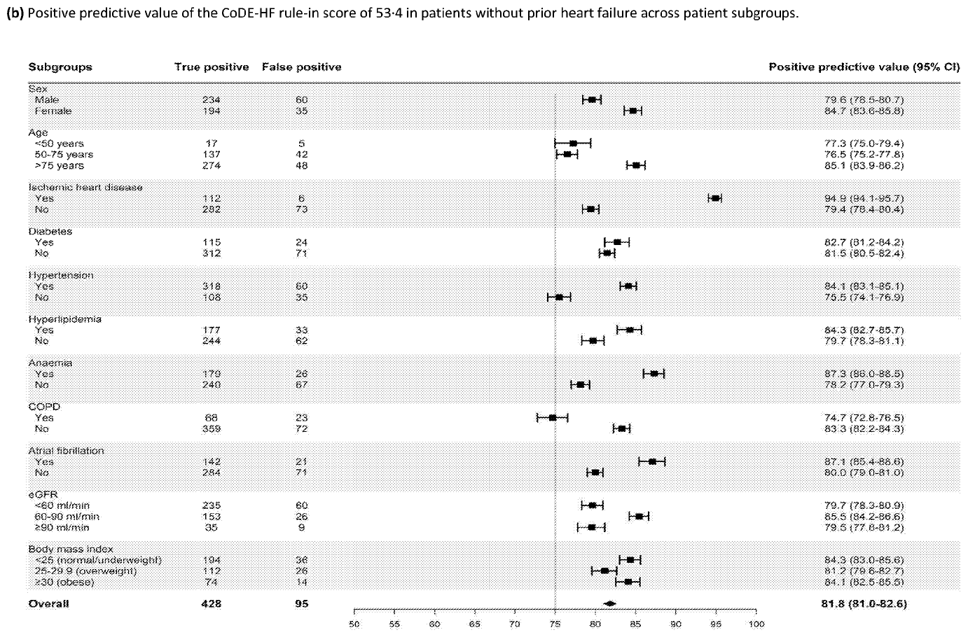

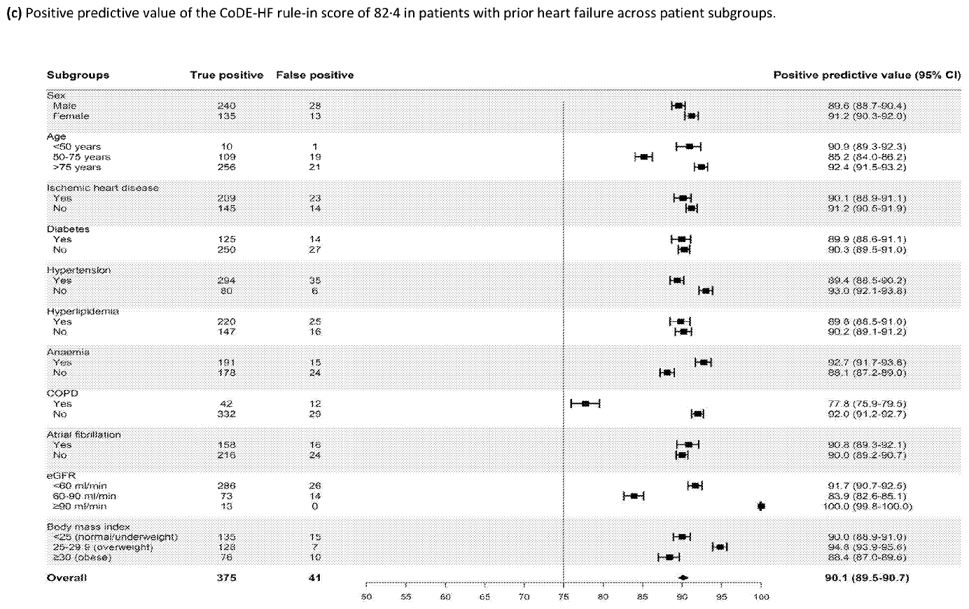

FIG. 41 illustrates Diagnostic performance of the CoDE-HF score across patient subgroups in the external validation cohort.

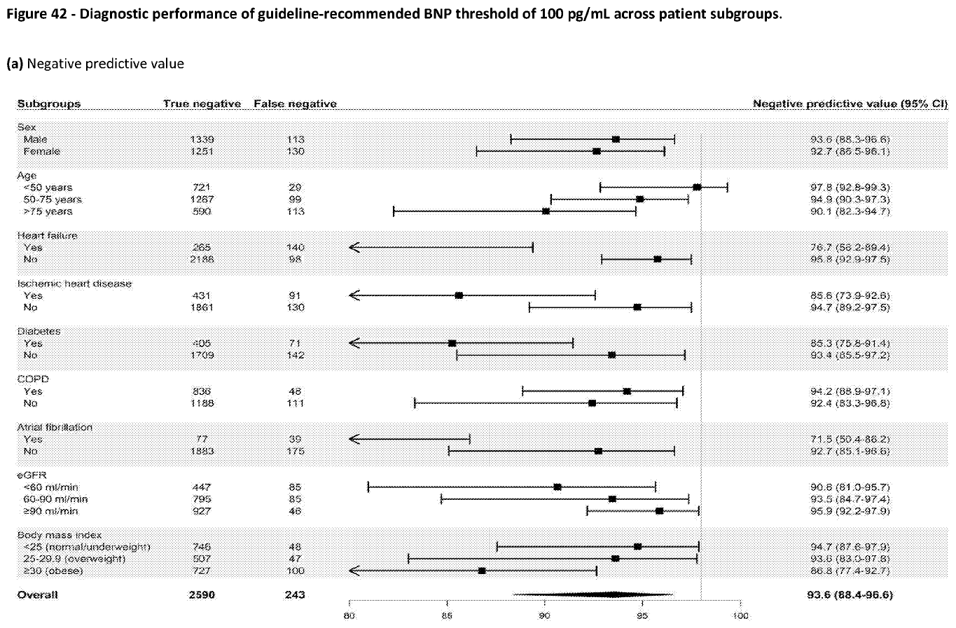

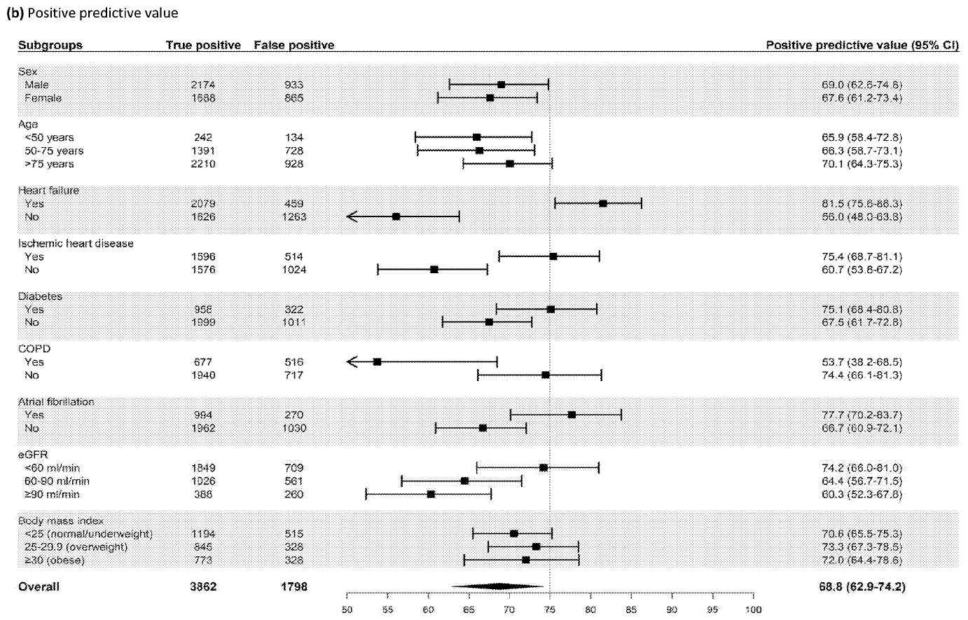

FIG. 42 illustrates Diagnostic performance of guideline-recommended BNP threshold of 100 pg/mL across patient subgroups.

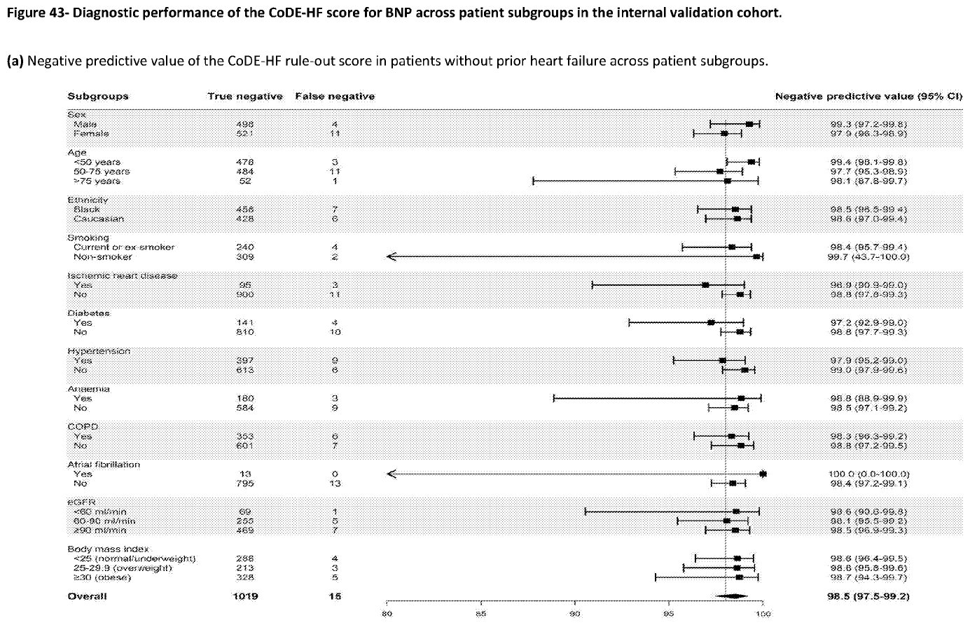

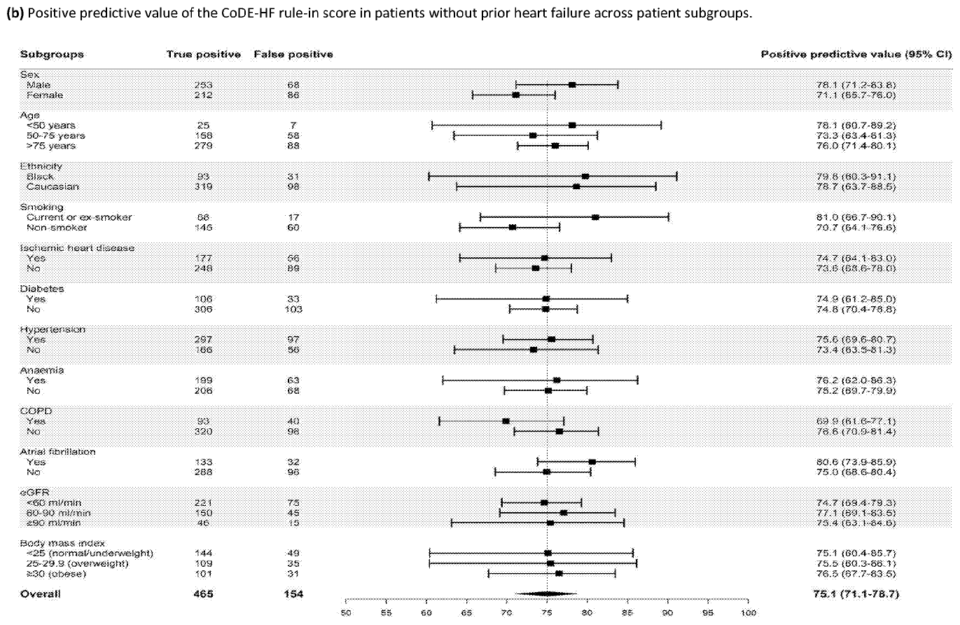

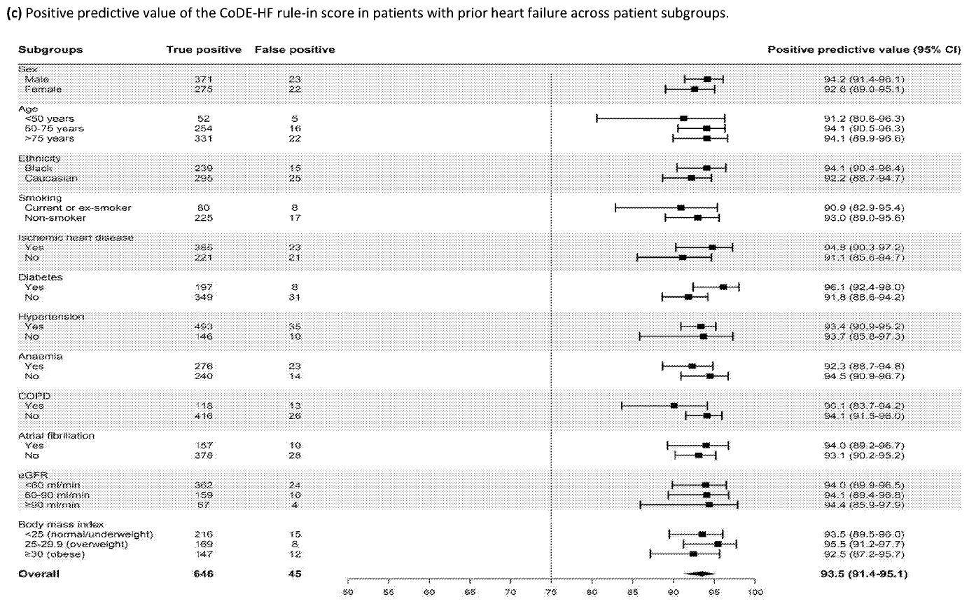

FIG. 43 illustrates Diagnostic performance of the CoDE-HF score for BNP across patient subgroups in the internal validation cohort.

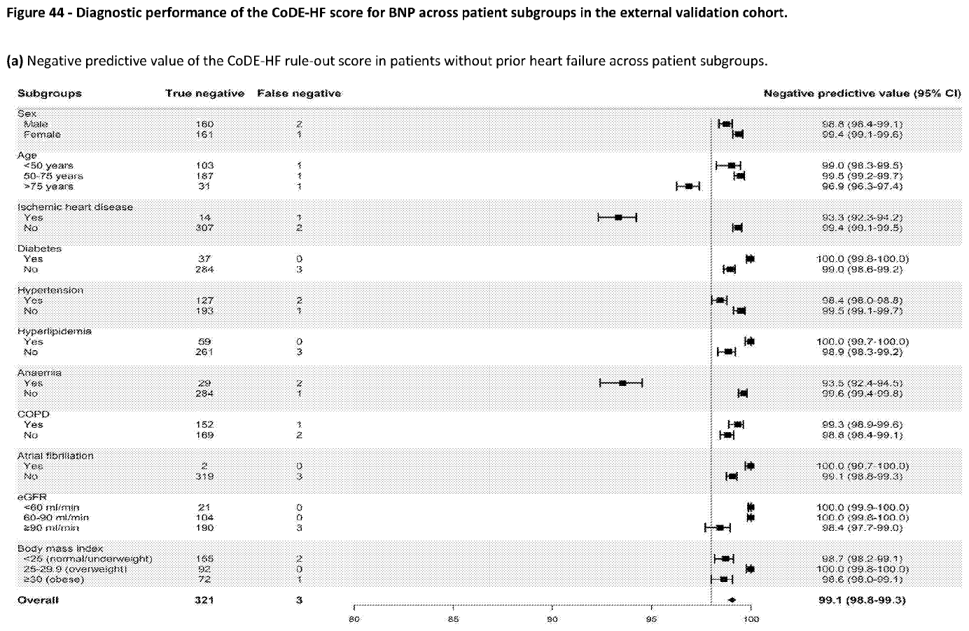

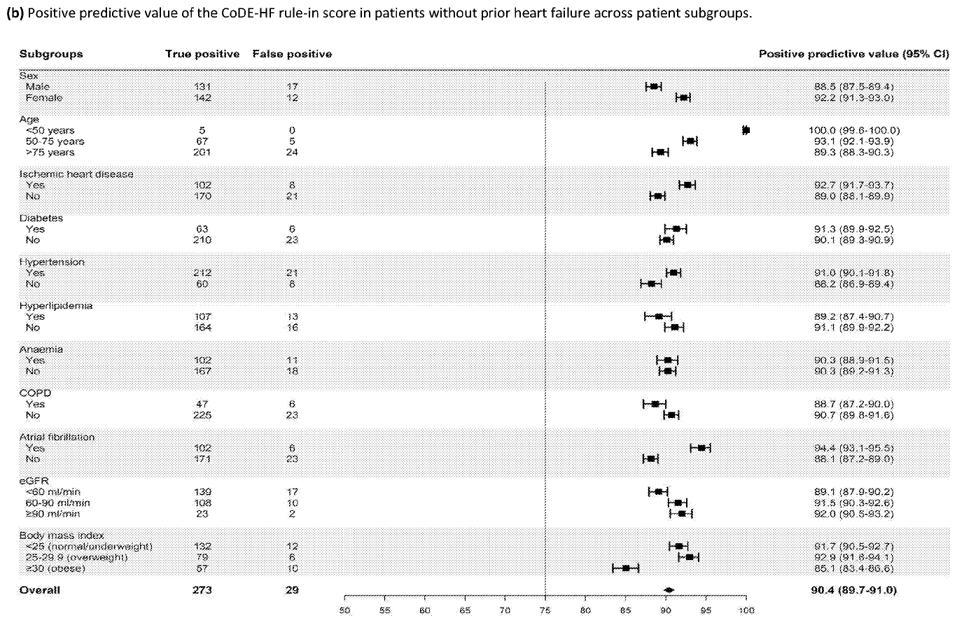

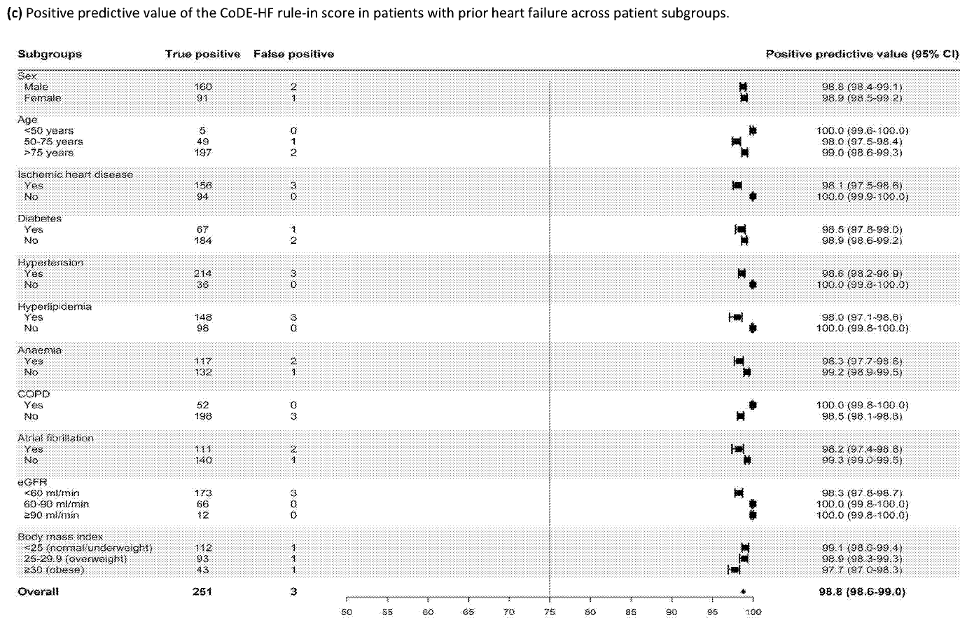

FIG. 44 illustrates Diagnostic performance of the CoDE-HF score for BNP across patient subgroups in the external validation cohort.

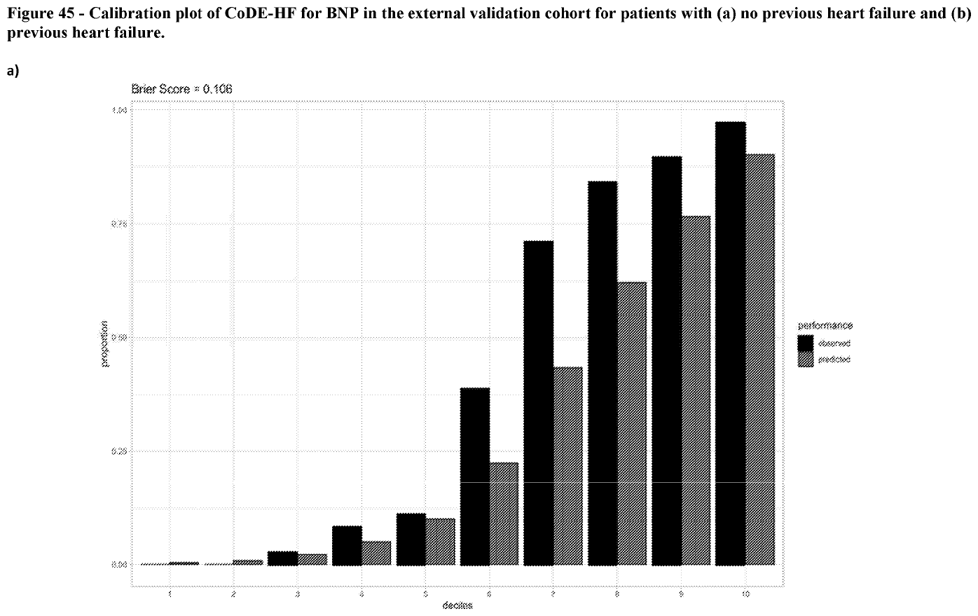

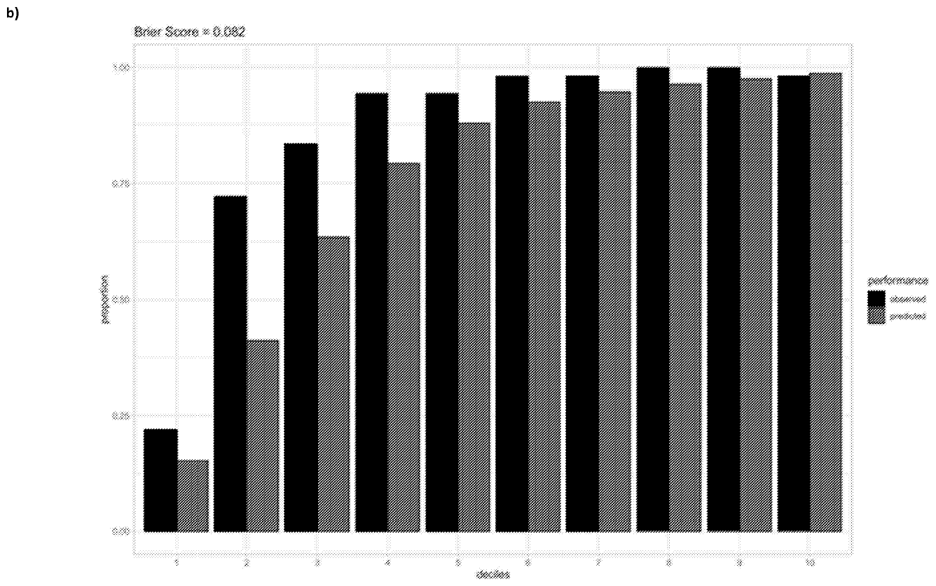

FIG. 45 illustrates Calibration plot of CoDE-HF for BNP in the external validation cohort for patients with (a) no previous heart failure and (b) previous heart failure.

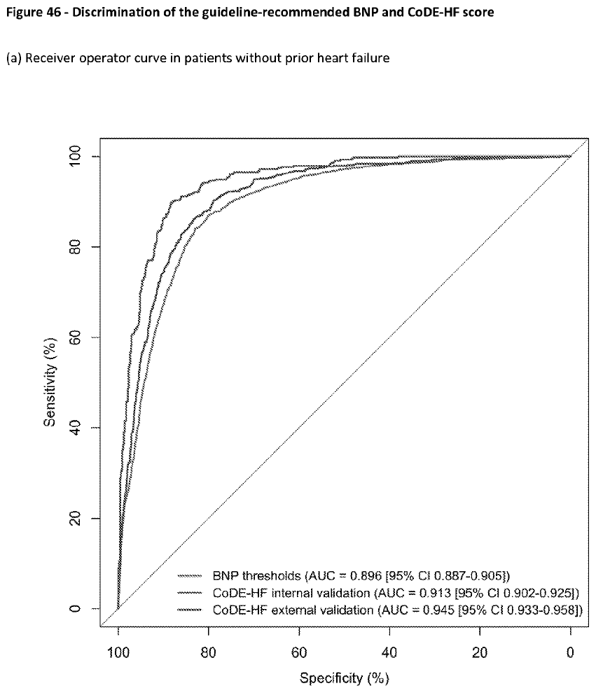

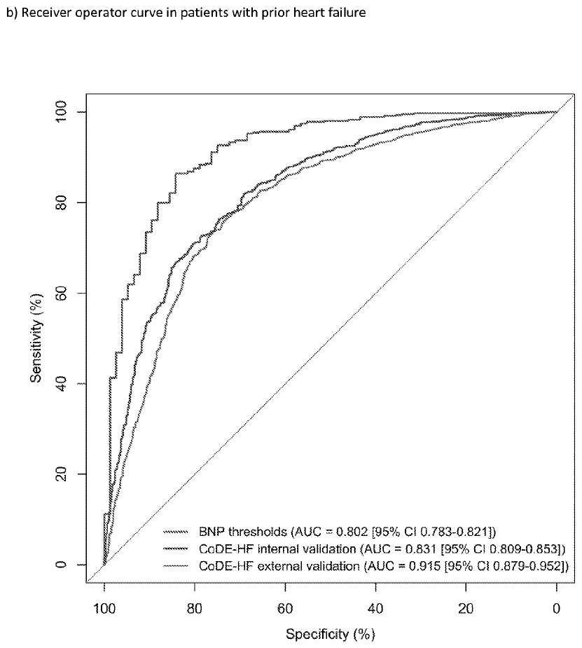

FIG. 46 illustrates Discrimination of the guideline-recommended BNP and CoDE-HF score

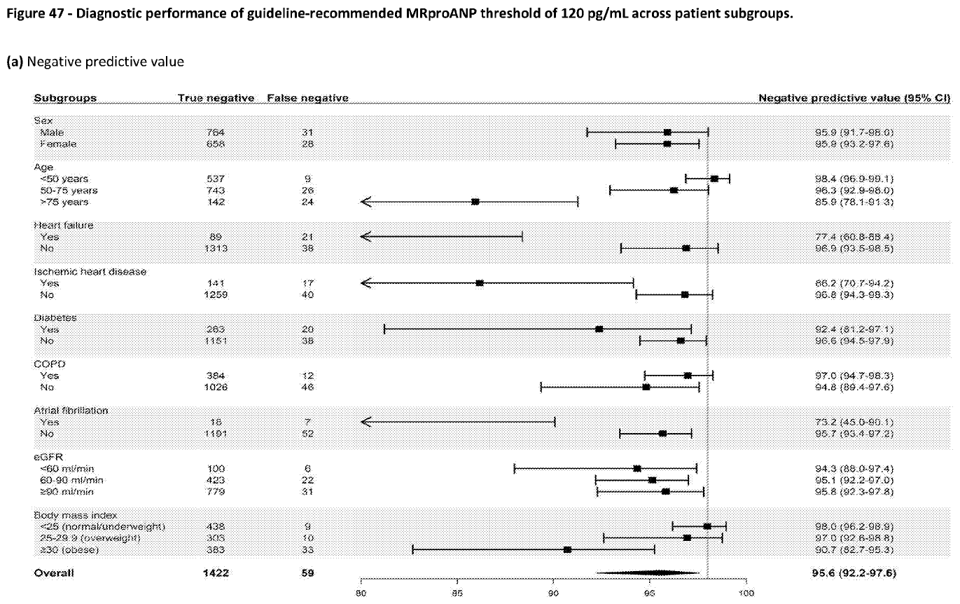

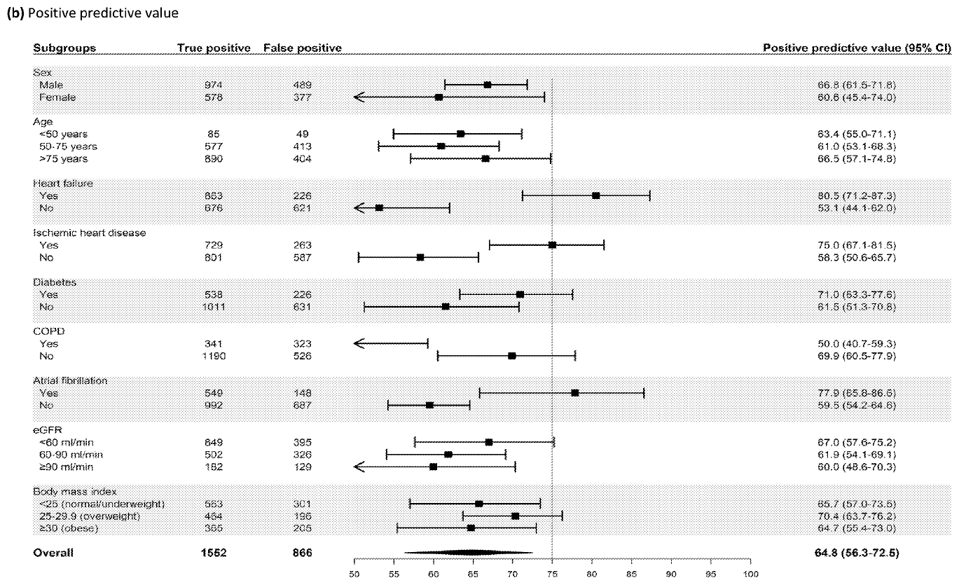

FIG. 47 illustrates Diagnostic performance of guideline-recommended MRproANP threshold of 120 pg/mL across patient subgroups.

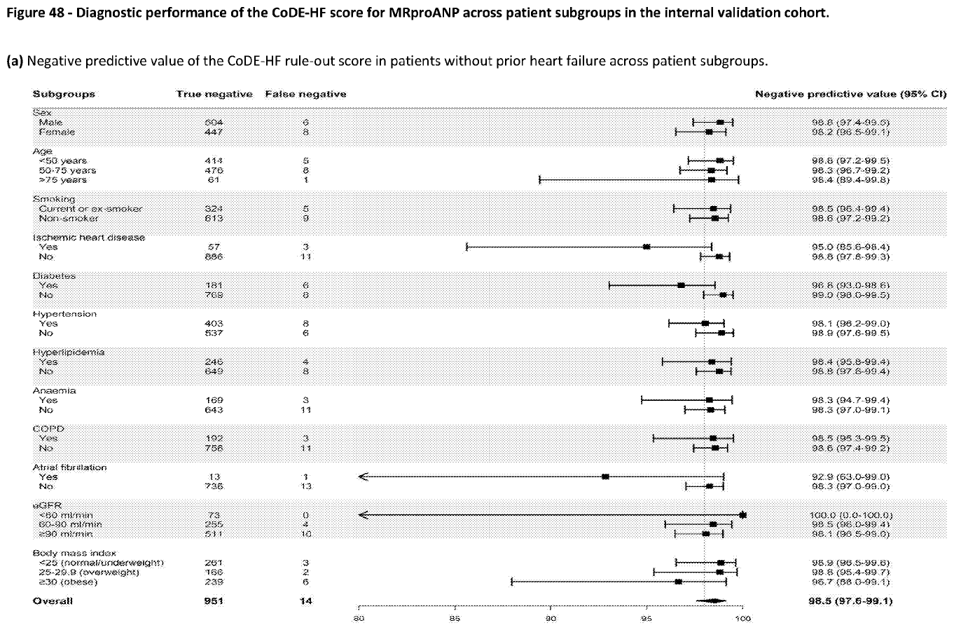

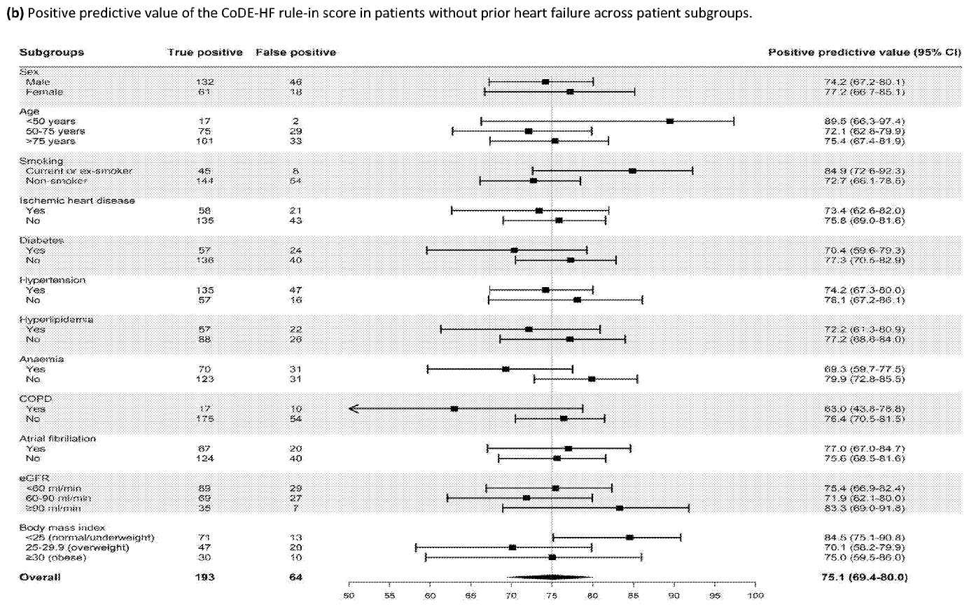

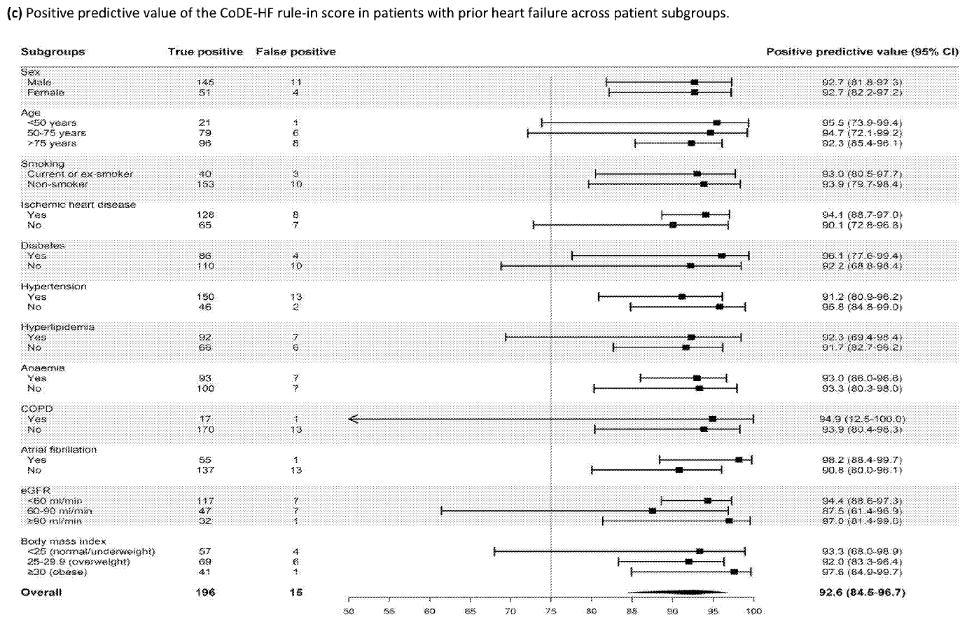

FIG. 48 illustrates Diagnostic performance of the CoDE-HF score for MRproANP across patient subgroups in the internal validation cohort.

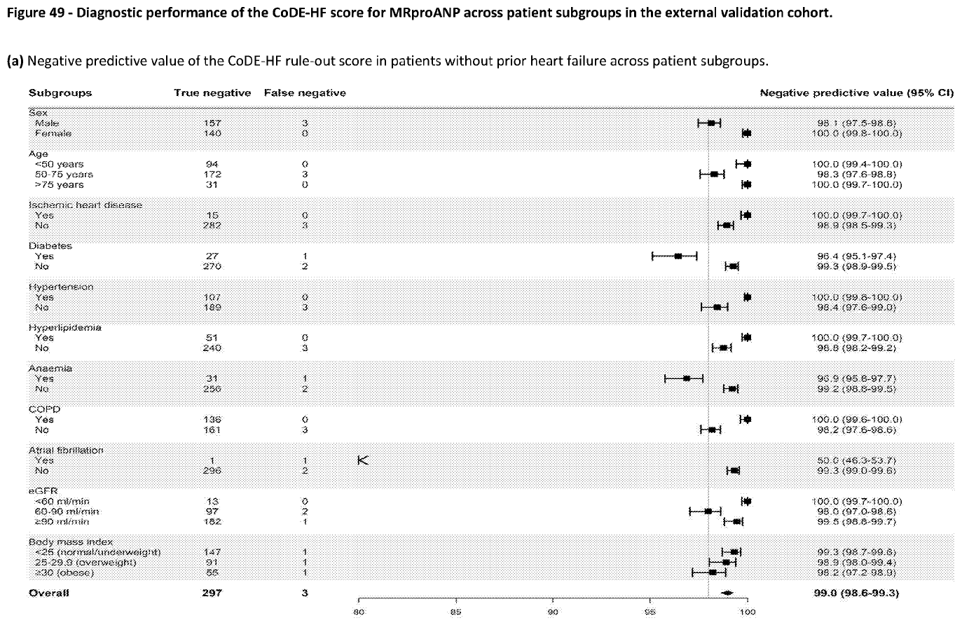

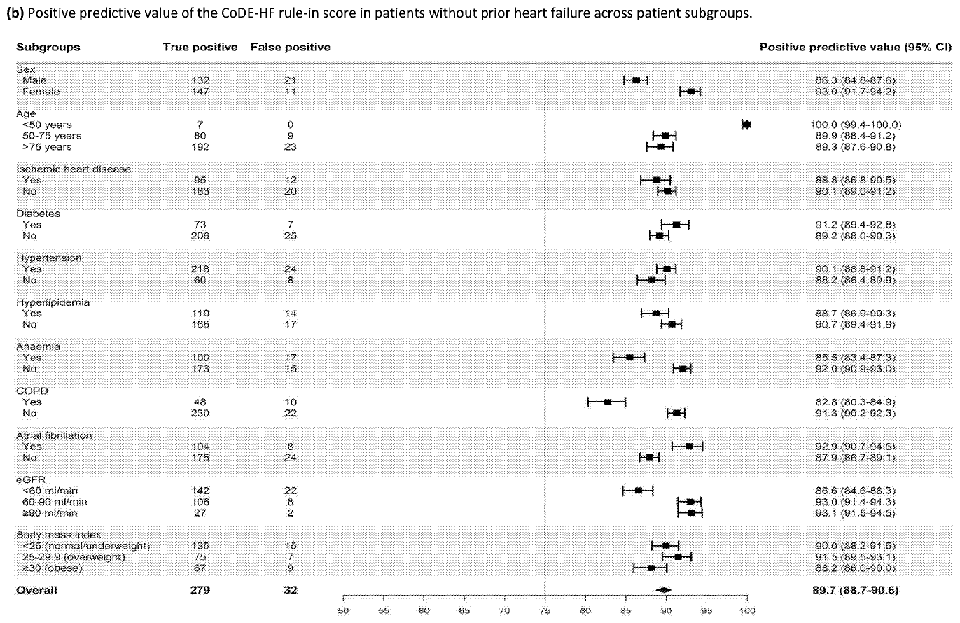

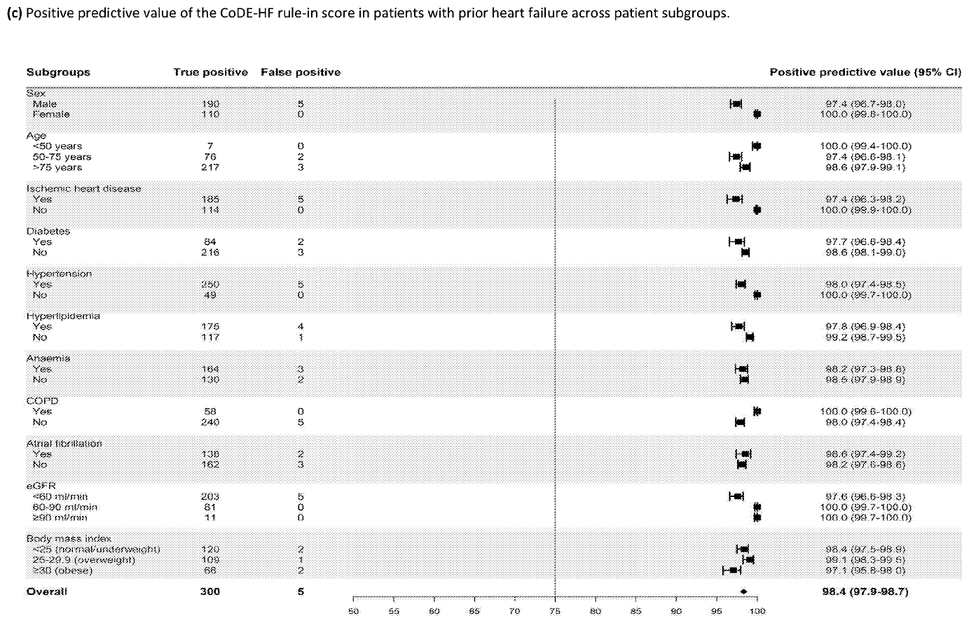

FIG. 49 illustrates Diagnostic performance of the CoDE-HF score for MRproANP across patient subgroups in the external validation cohort.

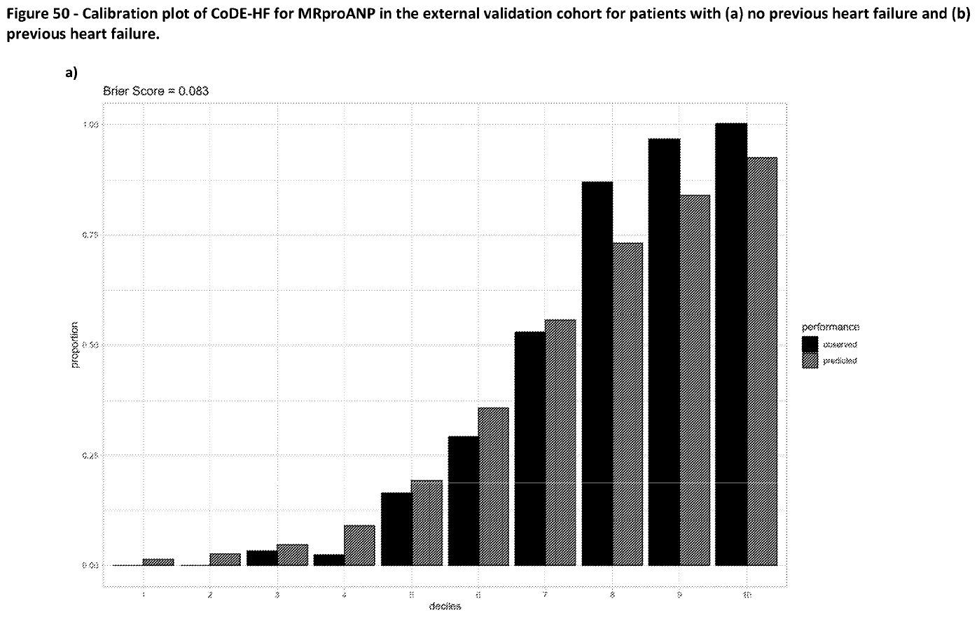

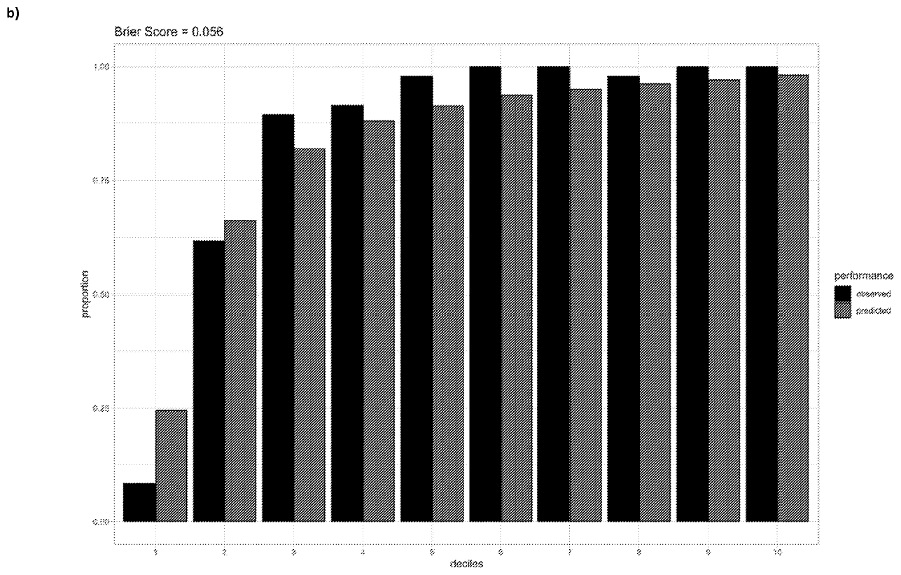

FIG. 50 illustrates Calibration plot of CoDE-HF for MRproANP in the external validation cohort for patients with (a) no previous heart failure and (b) previous heart failure.

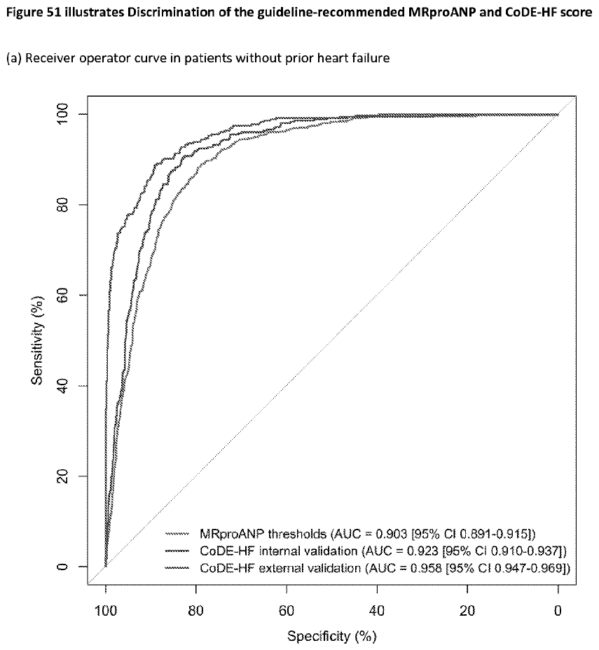

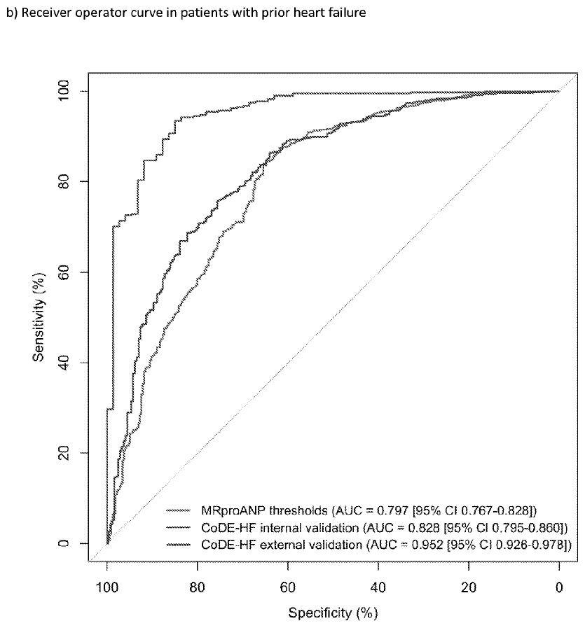

FIG. 51 illustrates Discrimination of the guideline-recommended MRproANP and CoDE-HF score

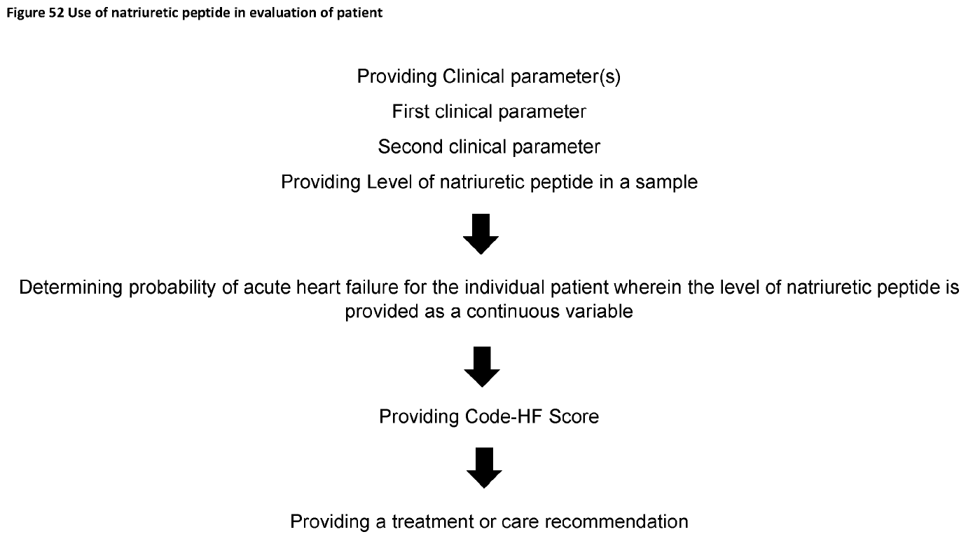

FIG. 52 illustrates flow diagram of method of the invention.

### DEFINITIONS

Heart failure is a condition in which the heart does not pump enough blood to meet the needs of the body. It is caused by dysfunction of the heart due to muscle damage (systolic or diastolic dysfunction), valvular dysfunction, arrhythmias or other rare causes. Acute heart failure can present as new-onset heart failure in people without known cardiac dysfunction, or as acute decompensation of chronic heart failure.

This is a life-threatening medical condition that requires urgent evaluation and treatment, typically leading to urgent hospital admission.

### DETAILED DESCRIPTION

Embase, Medline and Cochrane central register of controlled trials were searched for studies evaluating NT-proBNP in patients with suspected acute heart failure. Individual patient-level data was requested and diagnostic performance for the guideline-recommended rule-out (300 pg/mL) and age-specific rule-in (450, 900 and 1,800 pg/mL) thresholds were evaluated with random-effects meta-analysis. This provided fourteen studies from 13 countries which provided individual patient-level data in 10,365 patients, of which, 43.9% (4,549/10,365) had an adjudicated diagnosis of acute heart failure.

Meta-estimates of the sensitivity, specificity, negative predictive value (NPV) and positive predictive value (PPV) of the guideline-recommended NT-proBNP rule-out threshold (300 pg/mL) and age-specific rule-in thresholds (450, 900, and 1,800 pg/mL for those <50 years, 50-75 years, and >75 years respectively) for acute heart failure were derived using a two-stage approach, with estimates calculated separately within each study utilised, then pooled across studies by random effects meta-analysis.

At the rule-out threshold, the negative predictive value (NPV) was 94.6% (91.9%-96.4%), with significant heterogeneity across patient subgroups (FIG. 1). At the rule-in thresholds, the positive predictive values (PPV) for those <50 years, 50-75 years, and >75 years were 61.0% (55.3%-66.4%), 72.7% (62.1%-81.3%) and 80.5% (71.1%-87.4%), respectively.

Using the same approach, the inventors subsequently evaluated the diagnostic performance of NT-proBNP concentrations across a range of concentrations to determine a rule-out threshold that would identify the highest proportion of patients as low-probability for an NPV at or above 98% and a rule-in threshold that would identify the highest proportion of patients as high-probability for a PPV at or above 75%. This was then utilised with a generalized linear mixed model (GLMM) to compute a value (0-100) that would correspond to an individual patient's estimated probability of acute heart failure.

In patients without prior heart failure, the inventors model had good discrimination and calibration (area under the curve of 0.931 [0.925-0.938], Brier score of 0.094). A score of <5.6 and ≥45.2 identified 42.3% of patients as low-probability of acute heart failure (NPV 98.5%, 97.6%-99.1%) and 30.5% as high-probability (PPV 75.1%, 67.7%-81.3%) with consistent performance across subgroups.

In contrast to conventional techniques which utilise NT-proBNP concentrations at distinct values, the inventors considered this continuous measure of NT-proBNP and predefined simple and objective clinical variables that are known to be associated with acute heart failure (such as age, estimated glomerular filtration rate, hemoglobin, body mass index, heart rate, blood pressure, peripheral edema, prior history of heart failure, chronic obstructive pulmonary disease and ischemic heart disease) with the study identifier included as a random effects variable.

To account for any missing data across studies, the inventors multiply imputed ten datasets using joint-modelling multiple imputation with random study specific covariance matrices fitted with a Markov chain Monte Carlo algorithm. Due to the positive-skew in NT-proBNP concentrations, the inventors used a logarithmic transformation of NT-proBNP concentrations in the model. Further, they evaluated non-linear relationships between continuous variables and the diagnosis using multivariable fractional polynomial methods. Ten iterations of 10-fold cross-validation were used to generate the score for each patient. This score was then considered to identify the score that relative to an index value would classify the highest proportion of patients as high- or low-probability of acute heart failure with optimal performance to rule-in (75% PPV and 90% specificity) and rule-out (98% NPV and 90% sensitivity) acute heart failure. In addition, the inventors performed internal-external cross-validation to evaluate the performance of the model in each study. In brief, this approach iteratively leaves one study out at a time for external validation and uses the remaining studies for model development. In addition to GLMM, the inventors developed an extreme gradient boosting machine learning algorithm (XGBoost) using the same variables and cross-validation approach EXtreme Gradient Boosting (XGBoost) is a supervised machine learning technique proposed by Chen and Guestrin. In brief, gradient boosting employs an ensemble technique to iteratively improve model accuracy for regression and classification problems. This is achieved by creating sequential models, using decision trees as learners where subsequent models attempt to correct errors of the preceding models. XGBoost refers to the re-engineering of gradient boosting to significantly improve the speed of the algorithm by pushing the limits of computational resources.

The mathematical formula for the gradient boosting model can be described as:

$${\hat{y}}_{i} = {\sum }_{k = 1}^{K} {f}_{k} ( {x}_{i} ) , {f}_{k} ∈ F \qquad ( 1 )$$

where f is an function that map each variable vector xᵢ (xᵢ={xᵢ, x₂, . . . , xₙ}, i=1, 2, N) to the outcome yᵢ, K is the number of Classification and Regression Trees (CART) and F is the space of function containing all CART.

XGBoost optimises an objective function of the form:

$$Obj = {\sum }_{i = 1}^{N} l ( {y}_{i} , {\hat{y}}_{i} ) + {\sum }_{k = 1}^{K} Ω ( {f}_{k} ) \qquad ( 2 )$$

Where the first term is a loss function, l, which evaluates how well the model fits the data by measuring the difference between the prediction ŷᵢ and the outcome yᵢ. The second term, the regularization term, is used by XGBoost to avoid overfitting by penalizing the complexity of the model. Furthermore, to improve and fully leverage the advantages of XGBoost the inventors tuned the hyper-parameters of the algorithm through a grid search strategy using 10-fold cross-validation.

The hyper-parameter values for the model in patients without prior heart failure were: the number of iterations (trees) was set to 154, the learning rate (shrinkage parameter applied to each tree in the expansion) was set to 0.08, the interaction depth (maximum depth of each tree, expresses the highest level of variable interactions allowed) was set to 5, the minimum number of observations in the terminal nodes was set to 1, the fraction of the training set observations randomly selected for each subsequent tree was set to 0.94 and the fraction of variables randomly sampled for each tree was set to 0.58.

The hyper-parameter values for the model in patients with prior heart failure were: the number of iterations (trees) was set to 137, the learning rate (shrinkage parameter applied to each tree in the expansion) was set to 0.04, the interaction depth (maximum depth of each tree, expresses the highest level of variable interactions allowed) was set to 3, the minimum number of observations in the terminal nodes was set to 5, the fraction of the training set observations randomly selected for each subsequent tree was set to 0.88 and the fraction of variables randomly sampled for each tree was set to 0.74. As discussed herein, using the GLMM and XGBoost models as generated using the approach discussed, the score using the continuous variable of the natriuretic peptide measurement and at least two other clinical parameters was calculated and then considered.

Guideline-Recommended and Age-Specific NT-proBNP Thresholds

Pooled meta-estimates of NPV, sensitivity, PPV and specificity of NT-proBNP for the overall population at the guideline recommended rule-out threshold of 300 pg/mL were 94.6% (95% confidence interval, 91.9-96.4%), 96.8% (94.6-98.1%), 62.9% (51.3-73.3%), and 49.3% (35.3-63.4%) respectively (FIG. 5). Overall, 30.3% of patients with suspected acute heart failure had NT-proBNP below 300 pg/mL. However, there was significant heterogeneity across prespecified patient subgroups and across cohorts (FIG. 2 and FIGS. 6 and 7). NPV was lower in patients 75 years (88.2% [83.5-91.8%]), those with prior heart failure (79.4% [68.4-87.3%]), and obesity (90.3% [84.4-94.2%]).

Pooled meta-estimates of the PPV for age-specific NT-proBNP rule-in thresholds of 450, 900 and 1800 pg/mL were 61.0% (55.3-66.4%), 72.7% (62.1-81.3%) and 80.5% (71.1-87.4%), respectively. Corresponding specificities were 87.7% (79.3-93.0%), 81.1% (72.8-87.3%) and 73.8% (66.0-80.4%). Overall, 48.7% of patients with suspected acute heart failure had NT-proBNP above these age-specific thresholds. The PPV of the age-specific rule-in thresholds were higher than the uniform 300 pg/mL threshold in subgroups although there was heterogeneity across different age groups and renal function and across cohorts with differing prevalence of acute heart failure (FIGS. 8 to 13). In sensitivity analyses restricted to studies where adjudication of acute heart failure was blinded to NT-proBNP concentrations, the diagnostic performance of the guideline-recommended and age-specific NT-proBNP thresholds remained unchanged.

Optimized NT-proBNP Thresholds

An NT-proBNP threshold of 100 pg/mL achieved an optimal rule-out criteria with a pooled NPV of 97.8% (95.8-98.8%) and sensitivity of 99.3% (98.5-99.7%) (FIG. 3). However, NPV remains lower in older patients and those with a past medical history of heart failure, ischemic heart disease and impaired renal function (FIG. 14). Similarly, an NT-proBNP threshold of 1000 pg/mL achieved an optimal rule-in criteria with a PPV of 74.9% (64.4-83.2%) and specificity of 76.1% (65.6-84.2%), however performance was also lower within patient subgroups, particularly in those without prior heart failure (FIG. 3 and FIGS. 15 to 17).

The CoDE-HF Score

Due to differences in comorbidities and the prevalence of acute heart failure, models were developed and validated for patients with and without prior heart failure separately. Both GLMM and XGBoost models were well calibrated with excellent or outstanding discrimination between those with and without acute heart failure. The GLMM model used to derive the CoDE-HF score had AUCs of 0.931 (95% CI, 0.925-0.938) and 0.863 (0.848-0.878), and Brier scores of 0.094 and 0.121 for those without and with prior heart failure respectively (FIGS. 18 and 19).

Whereas conventionally diagnostic tests are binary in fashion —positive versus negative —in the present invention, the biomarkers, in particular natriuretic peptide, are provided as a continuous measure to make more individualised decisions and applied in the diagnosis of acute heart failure. Unlike previous tests, NTproBNP is provided in the model as a continuous variable, not merely as an elevated or otherwise parameter (ie. binary variable).

In patients without prior heart failure, a CoDE-HF score of 5.7 (95% Cl 5.5-5.9) achieved our target rule-out criteria with a NPV of 98.5% (97.6-99.1%) and sensitivity of 97.9% (96.1-98.9%) (FIG. 4), whilst a score of 45.2 (95% Cl 44.7-45.9) achieved the target rule-in criteria with a PPV of 75.1% (67.6-81.3%) and a specificity of 90.6% (85.8-93.9%). These rule-in and rule-out scores had similar diagnostic performance across all prespecified subgroups (FIGS. 20 to 25). If these scores were applied in patients with suspected acute heart failure, the score determined by the inventors would identify 42.3% at low-probability (<5.7) and 30.5% at high-probability (45.2) of acute heart failure. In patients with prior heart failure, there was no score which achieved the target rule-out criteria with either model. Using the method of the present invention a score of 86.1 (95% Cl 85.7-86.9) achieved a target rule-in criteria with a PPV of 93.2% (89.4%-95.6%) and specificity of 90.0% (82.1%-94.6%). This threshold would identify 47.9% of patients as high-probability for acute heart failure. Internal-external cross-validation using weighted average intercepts demonstrated excellent performance across all studies (FIGS. 26 to 32).

1. A method of identifying an individual's likelihood of having acute heart failure comprising the steps of combining the level of natriuretic peptide in a sample from an individual with at least two other clinical parameters from the individual in a statistical model to compute the probability of acute heart failure for the individual patient wherein the level of natriuretic peptide is provided as a continuous variable in the model.

2. A method of identifying an individual's likelihood of having acute heart failure as claimed in claim 1 wherein the statistical model is generated by a generalised linear mixed model [GLMM] or extreme gradient boosting machine learning algorithm [XGBoost].

3. The method of claim 1 wherein the clinical parameters are at least two parameters selected from the list comprising age, renal function, haemoglobin, body mass index, heart rate, blood pressure, for example systolic blood pressure, diastolic blood pressure, and/or mean arterial pressure, ECG data, cardiac biomarker concentration, peripheral oedema, prior history of heart failure, chronic obstructive pulmonary disease, ischaemic heart disease and diabetes mellitus.

4. A system to identify an individual's likelihood of having acute heart failure, the system comprising a computer processor, memory comprising one or more computer programs wherein one or more of the computer programs comprise a statistical model to compute the probability of acute heart failure for an individual patient by combining the level of natriuretic peptide in a sample from an individual with at least two other clinical parameters from the individual, optionally wherein the statistical model is generated by a generalised linear mixed model [GLMM] or extreme gradient boosting machine learning algorithm [XGBoost].

5. The method of claim 1 wherein the statistical model is generated extreme gradient boosting machine learning algorithm [XGBoost].

6. The method of claim 1 wherein a logarithmic transformation of a natriuretic peptide level is used in the statistical model.

7. The method of claim 6 wherein the natriuretic peptide level and clinical parameters are entered into the statistical model to generate the probability score for each patient and the probability score is assessed in individuals attending hospital due to suspected acute heart failure.

8. The method of claim 7 wherein the natriuretic peptide level and clinical parameters are entered into a statistical model to generate the probability score for each patient that would classify the highest proportion of patients as high- or low-probability of acute heart failure to rule-in and rule-out acute heart failure.

9. A method of identifying an individual's likelihood of having acute heart failure comprising the steps of
(a) obtaining the level of natriuretic peptide in a sample from the individual and
(b) obtaining values for least two other factors selected from a list comprising
age, renal function, haemoglobin, body mass index, heart rate, blood pressure, for example systolic blood pressure, diastolic blood pressure, and/or mean arterial pressure, ECG data, cardiac biomarker concentration, peripheral oedema, prior history of heart failure, chronic obstructive pulmonary disease, ischaemic heart disease and diabetes mellitus and assigning a probability of acute heart failure to the individual based on a statistical model generated such that the probability score for each patient of the model is provided to classify the highest proportion of the patients of the model as high- or low-probability of acute heart failure to rule-in and rule-out acute heart failure.

10. The method of claim 9 wherein the natriuretic peptide is selected from the group consisting of atrial natriuretic peptide (“ANP”), proANP, NT-proANP, B-type natriuretic peptide (“BNP”), NT-pro BNP, pro-BNP, mid-regional pro-atrial natriuretic peptide (MR-proANP), and C-type natriuretic peptide.

11. The method of claim 10 wherein the natriuretic peptide is selected from BNP, NT-pro BNP, mid-regional pro-atrial natriuretic peptide (MR-proANP) and pro-BNP.

12. The method of claim 11 wherein the natriuretic peptide is NT-pro BNP.

13. The method of claim 9 wherein renal function is measured by estimated glomerular filtration rate, creatinine clearance rate or serum/plasma creatinine.

14. The method of claim 9 wherein body mass index is represented by the use of two or more categories of underweight, normal weight, overweight or obese.

15. The method of claim 9 wherein the level of natriuretic peptide and the clinical parameters are assessed at a single point in time.

16. The method of claim 9 wherein the method further comprises providing a treatment or care recommendation.

17. The method of claim 9 wherein the method further comprises providing a treatment or care recommendation and the treatment or care recommendation is provided by a signal to a device.

18. The method of claim 17 wherein the signal is a visual signal to a display.

19. The method of claim 17 wherein the signal is a visual signal to a display on a portable device.

20. A computer based tool capable of receiving data to allow establishment or the ruling out of acute heart failure and a processor capable of providing a method of claim 9.

21. The computer based tool of claim 20 capable of providing a signal indicative of the status of acute heart failure in an individual.
